SEQUENCE LISTING (37 sequences)

SEQ ID NO: 1 - DNA, 19 bp, Artificial Sequence
  human Pit-1b (Oligo B)
gcctgaagtatctctcctc

SEQ ID NO: 2 - DNA, 64 bp, Artificial Sequence
  human Pit-1b (assembly oligo B1)
gatccccgcctgaagtatctctcctcttcaagagagaggagagagacttcaggctttttggaaa

SEQ ID NO: 3 - DNA, 64 bp, Artificial Sequence
  human Pit-1b (assembly oligo B2)
agcttttccaaaaagcctgaagtctctctcctctctcttgaagaggagagatacttcaggcggg

SEQ ID NO: 4 - DNA, 19 bp, Artificial Sequence
  human Pit-1a (Oligo A)
tatgtggcatacctctgga

SEQ ID NO: 5 - DNA, 64 bp, Artificial Sequence
  human Pit-1a (assembly oligo A1)
gatcccctatgtggcatacctctggattcaagagatccagaggcatgccacatatttttggaaa

SEQ ID NO: 6 - DNA, 64 bp, Artificial Sequence
  human Pit-1a (assembly oligo A2)
agcttttccaaaaatatgtggcatgcctctggatctcttgaatccagaggtatgccacataggg

SEQ ID NO: 7 - DNA, 19 bp, Artificial Sequence
  Pit-1c, control (Oligo C)
tccagaggtatgccacata

SEQ ID NO: 8 - DNA, 64 bp, Artificial Sequence
  Pit-1c (assembly oligo C1)
gatcccctatgtggcatgcctctggattcaagagatccagaggtatgccacatatttttggaaa

SEQ ID NO: 9 - DNA, 64 bp, Artificial Sequence
  Pit-1c (assembly oligo C2)
agcttttccaaaaatatgtggcatacctctggatctcttgaatccagaggcatgccacataggg

SEQ ID NO: 10 - DNA, 20 bp, Artificial Sequence
  Human Pit-1 forward
taccatcctcatctcggtgg

SEQ ID NO: 11 - DNA, 20 bp, Artificial Sequence
  Human Pit-1 reverse
tgacggcttgactgaactgg

SEQ ID NO: 12 - DNA, 410 bp, Homo  sapiens
  misc_feature (1)..(410): Human Pit-1 410 bp PCR fragment (corresponds to nt 1060-1469 of L20859)
taccatcctcatctcggtgggatgtgcagttttctgtgcccttatcgtctggttctttgtatgtcccaggatgaagagaaaaattgaacgagaaataaagtgtagtccttctgaaagccccttaatggaaaaaaagaatagcttgaaagaagaccatgaagaaacaaagttgtctgttggtgatattgaaaacaagcatcctgtttctgaggtagggcctgccactgtgcccctccaggctgtggtggaggagagaacagtctcattcaaacttggagatttggaggaagctccagagagagagaggcttcccagcgtggacttgaaagaggaaaccagcatagatagcaccgtgaatggtgcagtgcagttgcctaatgggaaccttgtccagttcagtcaagccgtca

SEQ ID NO: 13 - DNA, 20 bp, Artificial Sequence
  Human Pit-2 forward
tggatggtcattttgggttt

SEQ ID NO: 14 - DNA, 20 bp, Artificial Sequence
  Human Pit-2 reverse primer
gcacacctttggtaccgatt

SEQ ID NO: 15 - DNA, 385 bp, Homo  sapiens
  misc_feature (1)..(385): Human Pit-2 385 bp PCR fragment (corresponds to nt 265-649 of L20852)
tggatggtcattttgggtttcatcatagctttcatcttggccttttctgttggtgcaaacgatgttgccaactcctttggtacagccgtgggctctggtgtggtgaccttgaggcaggcatgcattttagcttcaatatttgaaaccaccggctccgtgttactaggcgccaaagtaggagaaaccattcgcaaaggtatcattgacgtgaacctgtacaacgagacggtggagactctcatggctggggaagttagtgccatggttggttccgctgtgtggcagctgattgcttccttcctgaggcttccaatctcaggaacgcactgcattgtgggttctactataggattctcactggtcgcaatcggtaccaaaggtgtgc

SEQ ID NO: 16 - DNA, 20 bp, Artificial Sequence
  Mouse Pit-1 forward
taccatcctcatctcggtgg

SEQ ID NO: 17 - DNA, 20 bp, Artificial Sequence
  Mouse Pit-1 reverse
tgacagtttgactgaactga

SEQ ID NO: 18 - DNA, 407 bp, Murine
  misc_feature (1)..(407): Mouse Pit-1 406 bp PCR fragment (corresponds to nt 1111-1517 of M73696)
taccatcctcatctcggtgggatgtgcagttttctgtgcccttatcgtctggttctttgtatgtcccaggatgaagagaaaaattgaacgagaagtaaagtctagtccgtctgaaagtcccttaatggaaaagaagagcaacttaaaagaagaccatgaagaaacaaagatggctcctggagacgttgagcataggaatcctgtgtctgaggtagtgtgtgccactgggccactccgggctgtggtggaggagaggacggtgtcattcaaacttggtgacctggaggaggctccggagcgagagcggcttcccatggacctgaaggaggagaccagcatagacagcaccatcaatggtgcagtgcagttgcctaatgggaaccttgttcagttcagtcaaactgtca

SEQ ID NO: 19 - DNA, 20 bp, Artificial Sequence
  Human sodium hydrogen antiporter (NHA) forward
gatccttctggcctgcctca

SEQ ID NO: 20 - DNA, 20 bp, Artificial Sequence
  Human sodium hydrogen antiporter (NHA) reverse primer
tcaggatggtgcccaggttt

SEQ ID NO: 21 - DNA, 248 bp, Homo  sapiens
  misc_feature (1)..(248): Human sodium hydrogen antiporter PCR fragment (corresponds to nt 377-624 of M81768)
gatccttctggcctgcctcatgaagataggtttccatgtgatccccactatctcaagcatcgtcccggagagctgcctgctgatcgtggtggggctgctggtggggggcctgatcaagggtgtaggcgagacaccccccttcctgcagtccgacgtcttcttcctcttcctgctgccgcccatcatcctggatgcgggctacttcctgccactgcggcagttcacagaaaacctgggcaccatcctga

SEQ ID NO: 22 - DNA, 27 bp, Artificial Sequence
  Full-length mouse Pit-1 forward primer
ggatccatggaatctactgtggcaacg

SEQ ID NO: 23 - DNA, 26 bp, Artificial Sequence
  Full-length mouse Pit-1 reverse primer
ctcgagtcacactggcaggatgatgt

SEQ ID NO: 24 - DNA, 2046 bp, Murine
  misc_feature (1)..(2046): Full length mouse Pit-1
atggaatctactgtggcaacgattactagtaccctagctgctgttactgcttccgctccaccgaagtatgacaatctatggatgctcatcctgggcttcatcattgcatttgtcttggcattctccgtgggagccaatgatgtagcaaattcgttcggtacagctgtaggctcaggtgtagtgaccctgaagcaagcctgcatcttagctagcatcttcgaaactgtgggctccgccttgctgggggccaaagtgagcgaaaccatccggaacggcttgatagatgtggagctgtacaacgaaactcaagatctgctcatggctggctccgtcagtgctatgtttggttctgctgtgtggcagctcgtggcttcgtttttgaagcttccgatttctgggacccattgtattgtcggtgcaaccattggtttctcccttgtggcaaatgggcagaagggtgtcaagtggtctgaactgataaaaattgtgatgtcgtggttcgtctctccgctgctttctggtattatgtctggaattttattcttccttgttcgtgcgttcatcctccgtaaggcagatccggttcctaatggcttacgagctttaccaattttttatgcctgcacaatcggaatcaacctcttttccattatgtatactggagcaccgttgctgggctttgacaaacttcctctgtggggtaccatcctcatctcggtgggatgtgcagttttctgtgcccttatcgtctggttctttgtatgtcccaggatgaagagaaaaattgaacgagaagtaaagtctagtccgtctgaaagtcccttaatggaaaagaagagcaacttaaaagaagaccatgaagaaacaaagatggctcctggagacgttgagcataggaatcctgtgtctgaggtagtgtgtgccactgggccactccgggctgtggtggaggagaggacggtgtcattcaaacttggtgacctggaggaggctccggagcgagagcggcttcccatggacctgaaggaggagaccagcatagacagcaccatcaatggtgcagtgcagttgcctaatgggaaccttgttcagttcagtcaaactgtcagcaaccagatcaactccagtggccactatcagtatcacaccgtgcacaaggattctggcttgtacaaggagctgctccataagttacatctggccaaggtgggagactgcatgggagattctggggacaagcccttgagacgcaacaacagctacacttcctacactatggcaatatgtggcatgcccctggattcattccgtgccaaagaaggtgaacaaaagggagatgaaatggagacgctgacatggcctaatgcagataccaagaagcggattcgaatggacagttacaccagttactgcaatgccgtgtctgaccttcactccgagtctgagatggacatgagtgtgaaggctgagatgggcctgggtgacagaaaaggaagcagtggctctcttgaagaatggtatgaccaggataagcctgaagtgtcccttctcttccagttcctgcagatccttacagcctgctttgggtcatttgcccatggtggcaatgacgtcagcaatgccatcggccctctggttgctttgtatcttgtttataaacaagaagcctctacaaaagcggcaacacccatatggcttctgctttatggtggtgttggcatttgcatgggcctgtgggtttggggaagaagagttatccagaccatggggaaggacctgaccccaatcacaccctccagtggtttcagtattgaactggcgtctgccttaactgtggtcatcgcatcaaacattggccttcccatcagcacaacacattgcaaagtgggctctgttgtgtctgttggctggctccgatcaaagaaggctgttgactggcgactgtttcgaaacatttttatggcctggtttgtcacggtccccatctctggggttatcagtgccgctatcatggcagtattcaagtacatcatcctgccagtgtga

SEQ ID NO: 25 - DNA, 22 bp, Artificial Sequence
  Human Pit-1 forward B
ggagggtgtcaagtggtctgaa

SEQ ID NO: 26 - DNA, 23 bp, Artificial Sequence
  Human Pit-1 reverse B
atctgccttatggaggatgaatg

SEQ ID NO: 27 - DNA, 24 bp, Artificial Sequence
  Human Pit-1 the probe B
ctgataaaaattgtgatgtcttgg

SEQ ID NO: 28 - DNA, 23 bp, Artificial Sequence
  Human Pit-2 forward B
tacaacgagacggtggagactct

SEQ ID NO: 29 - DNA, 20 bp, Artificial Sequence
  Human Pit-2 reverse B
aagcaatcagctgccacaca

SEQ ID NO: 30 - DNA, 15 bp, Artificial Sequence
  Human Pit-1 probe B
ccatggttggttccg

SEQ ID NO: 31 - DNA, 17 bp, Artificial Sequence
  Human Cbfa-1 forward
cccgtggccttcaaggt

SEQ ID NO: 32 - DNA, 24 bp, Artificial Sequence
  Human Cbfa-1 reverse
tgacagtaaccacagtcccatctg

SEQ ID NO: 33 - DNA, 15 bp, Artificial Sequence
  Human Cbfa-1 probe
agccctcggagaggt

SEQ ID NO: 34 - DNA, 25 bp, Artificial Sequence
  Human OPN forward
tgtcctctgaagaaaccaatgactt

SEQ ID NO: 35 - DNA, 23 bp, Artificial Sequence
  Human OPN reverse
tcatggctttcgttggacttact

SEQ ID NO: 36 - DNA, 17 bp, Artificial Sequence
  Human OPN probe
aaacaagagacccttcc

SEQ ID NO: 37 - protein, 40 aa, Artificial Sequence
  Pit-1 peptide
QAVVEERTVSFKLGDLEEAPERERLPSVDLKEETSIDSTV

(19) United States
(12) Patent Application Publication
(10) Pub. No.: US 2008/0280847 A1
(43) Pub. Date: Nov. 13, 2008

(54) PIT-1 AND VASCULAR CALCIFICATION

(75) Inventors: Cecilia Maria Giachelli, Mill Creek, WA (US); Xianwu Li, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: WASHINGTON, UNIVERSITY OF, Seattle, WA (US)

(21) Appl. No.: 12/023,923

(22) Filed: Jan. 31, 2008

Related U.S. Application Data
(60) Provisional application No. 60/894,077, filed on Mar. 9, 2007.

(51) Int. Cl. A61K 31/70; C07H 21/04; A61P 43/00; C12N 5/06

(52) U.S. Cl. 514/44; 536/24.5; 435/375

(57) ABSTRACT

The present invention relates to methods and compositions comprising small interfering RNAs (siRNAs) to identify nucleotides encoding proteins involved in the co-transport of inorganic phosphate in vascular tissues. Specifically, it relates to the identification of Pit-1 and Pit-2 as key proteins involved in vascular calcification, the deposition of calcium phosphate, in blood vessels. The nucleic acids encoding specific siRNAs and vectors encoding these siRNAs are useful as tools to specifically inhibit the expression of Pit-1, Pit-2, or related proteins in various primary and stably-transformed cell lines, and as tools to explore the roles and functional relationships of these proteins involved in vascular calcification. In addition, they may be useful directly as therapeutic agents in humans delivered to sites of vascular calcification.

### CROSS-REFERENCE TO RELATED APPLICATION

This application claims the benefit of U.S. Provisional Application No. 60/894,077, filed Mar. 9, 2007.

### STATEMENT OF GOVERNMENT LICENSE RIGHTS

This invention was made with U.S. Government support under NIH grant HL-62329 awarded by the National Institutes of Health, and the University of Washington Engineered Biomaterials (UWEB) Optical Microscopy and Image Analysis Shared Resource grants EEC-9872882 and EEC-9529161 awarded by the National Science Foundation. The U.S. Government has certain rights in the invention.

### FIELD OF THE INVENTION

The present invention relates to methods and compositions comprising small interfering RNAs (siRNAs) to identify nucleotides encoding proteins involved in the co-transport of inorganic phosphate in vascular tissues. Specifically, it relates to the identification of Pit-1 and Pit-2 as key proteins involved in vascular calcification, the deposition of calcium phosphate, in blood vessels. The nucleic acids encoding specific siRNAs and vectors encoding these siRNAs are useful as tools to specifically inhibit the expression of Pit-1, Pit-2, or related proteins in various primary and stably-transformed cell lines, and as tools to explore the roles and functional relationships of these proteins involved in vascular calcification. In addition, they may be useful directly as therapeutic agents in humans delivered to sites of vascular calcification.

### BACKGROUND OF THE INVENTION

Vascular calcification refers to calcium phosphate deposition in blood vessels, myocardium, and cardiac valves under pathological conditions. Vascular calcification is associated with increased risk of cardiovascular morbidity and mortality (Wilson et al., Circulation. 2001; 103: 1529-1534; Wayhs et al., J Am Coll Cardiol. 2002; 39: 225-230). It is highly prevalent in patients with atherosclerosis, diabetes, and renal failure (Christian R C, Fitzpatrick L A. Curr Opin Nephrol Hypertens. 1999; 8: 443-448). Approximately 50% of all deaths in end-stage renal disease (ESRD) patients are attributed to cardiovascular diseases (United States Renal Data System. USRDS 1999 Annual Data Report. Bethesda, Md.: National Institutes of Health, National Institute of Diabetes and Digestive and Kidney Disease; 1999. Available at: www.usrds.org/chapters/ch06.pdf. Accessed Dec. 10, 2002). Intimal, as well as medial, calcification contributes to the increased mortality in these patients (London et al., Nephrol Dial Transplant. 2003; 18: 1731-1740). Recent evidence indicates that vascular calcification is an actively regulated process, and molecules that inhibit or promote vascular calcification are currently being identified (Speer M Y, Giachelli C M. Cardiovasc Pathol. 2004; 13: 63-70; Giachelli C M. J Am Soc Nephrol. 2003; 14: S300-S304). Mineralization inhibiting molecules appear to play a major role in preventing ectopic calcification under physiological conditions. However, processes that promote calcification may predominate under pathological conditions and are less well defined.

Although the mechanisms of vascular calcification are not fully understood, abnormalities in mineral metabolism are considered important risk factors. In particular, phosphate has emerged as an important regulator of vascular calcification as well as a risk factor for cardiovascular mortality in dialysis patients. In humans, normal levels of serum phosphate range from 1 to 1.5 mmol/L (Alfrey et al., Adv Exp Med. Biol. 1978; 103: 187-193). Hyperphosphatemia, typically observed in patients with ESRD, is defined as serum phosphate levels exceeding 2 mmol/L. In these patients, hyperphosphatemia and elevated calcium×phosphate product are highly correlated with calcification of coronaries, peripheral arteries, and cardiac valves (Shigematsu et al., Nephrol Dial Transplant. 2003; 18 (suppl 3): iii86-iii89; London et al., J Am Soc Nephrol. 2000; 11: 778-783; Goodman et al., N Engl J. Med. 2000; 342: 1478-1483; Block et al., Am J Kidney Dis. 1998; 31: 607-617). Importantly, several large epidemiological studies have found that elevated serum phosphate is a major, nontraditional risk factor for cardiovascular mortality in ESRD patients (Block et al., Am J Kidney Dis. 1998; 31: 607-617; Raggi et al., J Am Coll Cardiol. 2002; 39: 695-701).

The mechanisms that drive vascular calcification are currently under intense investigation. It has been reported that elevated phosphate induces smooth muscle cell (SMC) calcification in vitro (Wada et al., Circ Res. 1999; 84: 166-178; Steitz et al., Circ Res. 2001; 89: 1147-1154; Chen et al., Kidney Int. 2002; 62: 1724-1731; Sugitani et al., Atheroscler Thromb. 2003; 10: 48-56). Concomitant with mineralization, elevated phosphate leads to SMC phenotypic change exemplified by upregulation of osteochondrogenic differentiation markers, such as Cbfa-1 and osteopontin (OPN) (Steitz et al., Circ Res. 2001; 89: 1147-1154; Chen et al., Kidney Int. 2002; 62: 1724-1731; Jono et al., Circ Res. 2000; 87: e10-e17). Subsequent studies implicated sodium-dependent phosphate cotransporters in phosphate-induced vascular calcification. Inhibition of sodium-dependent phosphate cotransporters by phosphonoformic acid blocked both phosphate-induced phenotypic transition and calcification in vascular SMC (Chen et al., Kidney Int. 2002; 62: 1724-1731; Jono et al., Circ Res. 2000; 87: e10-e17). These studies suggested that elevated phosphate, via a sodium-dependent phosphate cotransporter-mediated mechanism, might have direct pro-mineralizing effects on SMC independent of raising the calcium x phosphate product.

The types and mechanisms of sodium-dependent phosphate cotransporter function in vascular calcification are not currently known. Sodium-dependent phosphate cotransporters are glycosylated proteins with 7 to 10 transmembrane-spanning regions. The common function of sodium-dependent phosphate cotransporters is transporting phosphate from the extracellular environment into the cell in a sodium-dependent manner. Three types of sodium-dependent phosphate cotransporters have been identified based on structure, tissue expression, and regulation. The type I and type II sodium-dependent phosphate cotransporters are expressed predominantly in kidney and intestinal epithelium. The type I family includes NaPi-1, RNaPi-1, NPT-1, and Npt-1 and have been identified from various species (Werner et al., J Exp Biol. 1998; 201: 3135-3142; Takeda et al., Int J Biochem Cell Biol. 1999; 31: 377-381). The physiological roles of this family remain to be elucidated. In contrast, the members of the sodium-dependent phosphate cotransporters type II family, including NaPi-2, NaPi-3, NaPi-4, NaPi-5, NaPi-6, and NaPi-7, are crucially involved in renal and intestinal phosphate absorption and play an important role in the maintenance of serum phosphate homeostasis (Werner et al., J Exp Biol. 1998; 201: 3135-3142; Takeda et al., Int J Biochem Cell Biol. 1999; 31: 377-381). The type III sodium-dependent phosphate cotransporters, Pit-1 and Pit-2, are the most recently discovered subtypes and were originally identified as cell surface receptors for the gibbon ape leukemia virus (Glvr-1) and the amphotropic murine retrovirus (Ram-1), respectively (O'Hara et al., Cell Growth Differ. 1990; 1: 119-127; Miller et al., Proc Natl Acad Sci USA. 1994; 91: 78-82; Kavanaugh et al., Kidney Int. 1996; 49: 959-963). Type III sodium-dependent phosphate cotransporters are expressed in many tissues and cell types including kidney, brain, heart, liver, lung, osteoblast, and SMC, but their requirement in crucial cellular processes has not yet been determined. In the present studies, we have examined the role of the predominant phosphate cotransporter found in SMC, Pit-1, in phosphate-induced SMC calcification using RNA interference. We provide novel evidence that phosphate transport through Pit-1 plays a critical role in SMC calcification in vitro. In addition, our results further confirm that the modulation of SMC phenotypic change could be one of the mechanisms by which phosphate, via Pit-1, mediates calcification of vascular SMC cultures.

### SUMMARY OF THE INVENTION

Hyperphosphatemia is an important contributor to vascular calcification which is associated with cardiovascular morbidity and mortality. Earlier studies have demonstrated that elevated phosphate induces calcification of smooth muscle cells (SMC) in vitro (Steitz et al., Circ Res. 2001; 89: 1147-1154; Jono et al., Circ Res. 2000; 87: e10-e17). Inhibition of phosphate transport by phosphonoformic acid blocked phosphate-induced calcification, implicating sodium-dependent phosphate cotransporters in this process. In this study, the role of the type III sodium-dependent phosphate cotransporter, Pit-1, in SMC calcification in vitro was investigated by testing whether knockdown of Pit-1 expression by siRNA inhibited sodium-dependent phosphate uptake or phosphate-dependent SMC calcification. Human SMC stably expressing Pit-1 small interfering double-stranded RNA (SMC-iRNA) were established using a retroviral system. SMC-iRNA had decreased Pit-1 mRNA and protein levels and sodium-dependent phosphate transport activity compared with the control transduced cells (SMC-CT) (2.9 versus 9.78 nmol/mg protein per 30 minutes, respectively). Phosphate-induced SMC calcification was also significantly inhibited in SMC-iRNA compared with SMC-CT at all time points examined. Overexpression of Pit-1 restored phosphate uptake and phosphate-induced calcification in Pit-1 deficient cells, indicating that phosphate uptake is required for SMC calcification induced by elevated phosphate. Mechanistically, although Pit-1-mediated SMC calcification was not associated with apoptosis or cell-derived vesicles, inhibition of phosphate uptake in Pit-1 knockdown cells blocked the induction of the osteogenic markers Cbfa-1 and osteopontin. These results indicate that phosphate uptake through Pit-1 is essential for SMC calcification and phenotypic modulation in response to elevated phosphate. Our studies also suggest that Pit-1-dependent SMC calcification is not mediated by enhanced apoptosis or membrane-bound vesicle phosphate loading. Inhibition of phosphate uptake in Pit-1 knockdown cells, however, did abrogate induction of osteogenic markers, such as Cbfa-1 and OPN, in human SMC. These findings demonstrate a requirement for phosphate uptake via the sodium-dependent phosphate cotransporter, Pit-1, for SMC phenotypic transition and calcification that is likely to be important in the development of ectopic calcification of blood vessels under hyperphosphatemic conditions. These studies also point to Pit-1 as a potential target for therapies aimed at reducing vascular calcification.

The present invention relates to methods and compositions comprising small interfering RNAs (siRNAs) to identify nucleotides that encode proteins involved in the co-transport of inorganic phosphate in vascular tissues. More specifically, the invention relates to the identification of Pit-1 and Pit-2 as key proteins involved in vascular calcification, the deposition of calcium phosphate, in blood vessels. The nucleic acids encoding specific siRNAs and vectors encoding these siRNAs are useful as tools to specifically inhibit the expression of Pit-1, Pit-2, or related proteins in various primary and stably-transformed cell lines, and as tools to explore the roles and functional relationships of these proteins involved in vascular calcification, as noted below. In addition, they may be useful directly as human therapeutic agents delivered to sites of vascular calcification.

One embodiment of the invention relates to a double-stranded siRNA molecule that specifically down regulates expression of a Pit-1 gene via RNA interference wherein: (a) each strand of said siRNA is independently about 18 to about 28 nucleotides in length; and (b) one strand of said siRNA molecule comprises a target sequence having sufficient complementarity to an RNA of said Pit-1 gene for the siRNA molecule to direct cleavage of said RNA via RNA interference.

Another embodiment of the invention relates to a method of inhibiting expression of Pit-1 in primary, immortal, or transformed cells comprising administering to said cells siRNA molecules that direct cleavage of a target Pit-1 mRNA sequence present in said cells thereby effecting said inhibition.

### DEFINITIONS

The following is a list of terms and their definitions as used in the specification and the claims:

The term “primary cells” means cells that are derived directly from tissue (often embryonic tissue). They are distinct from immortal and transformed cells.

The term “immortal cells” means cells that can keep on dividing in the presence of growth factors. They are typically anchored to a solid support.

The term “transformed cells” means cells that can keep on dividing, but do not need growth factors for their growth. They typically can form colonies in the absence of a solid support.

Abbreviations and their corresponding meanings include:

bp=base pair(s)

ds=double-stranded

g=gram(s)

kb kilobase pair(s)

mg milligram(s)

ml or mL=milliliter(s)

mm=millimeter(s)

mM=millimolar

nt=nucleotide(s)

nmol=nanomole(s)

pmol=picomole(s)

ppm=parts per million

RT=room temperature

shRNA stem/hairpin RNA

siRNA small interfering RNA

SMC=smooth muscle cell(s)

U=units

ug or μg=microgram(s)

ul or uL or μl=microliter(s)

uM or μM=micromolar

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 sets forth the expression of Pit-1 and Pit-2 mRNA in human SMC. Panel A, Total RNA was isolated from human SMC; identical amounts of RNA were used for RT-PCR reaction with primers for human Pit-1, Pit-2, or GAPDH, respectively. A 409-bp fragment of Pit-1 and a 384-bp fragment of Pit-2 were detected. Panel B, Ten nanograms of total RNA were used for real-time PCRs for human Pit-1 or Pit-2, respectively. Data are expressed as mean ±SD from 3 individual PCR reactions: 29.1±2.03 and 3.52±0.131 for Pit-1 and Pit-2, respectively. *Significant decrease (P=0.004) compared with Pit-1.

FIG. 2 sets forth the specific knockdown of Pit-1 mRNA and protein levels by Pit-1 siRNA. Panel A, Northern blot analysis of RNA from cells stably expressing Pit-1 siRNA (SMC-iRNA) or control RNA (SMC-CT). The blots were hybridized with probes for human Pit-1, Pit-2, or NHA, respectively. mRNA levels were visualized by Phosphorlmager (Amersham Biosciences). Panel B, Quantitation of Pit-1, Pit-2, or NHA mRNA levels. The blots from Panel A were stripped and hybridized with an 18S ribosomal RNA probe. mRNA levels were normalized to 18S rRNA and data are presented as the percentage of SMC-CT levels. Panel C, Western blot analysis of Pit-1 protein. Protein lysates were prepared from SMC-iRNA and SMC-CT. Specific proteins were detected using anti-Pit-1 or anti-B-tubulin antibodies. Panel D, Pit-1 protein levels were quantified by densitometry and normalized to B-tubulin levels. Data are presented as the percentage of SMC-CT levels. Identical results were obtained in a replicate experiment. Panel 2E sets forth the effects of different siRNAs on Pit-1 expression in stably-transformed SMC cells, as noted in the general methods section below. Oligonucleotide B corresponds to hPit-1b (SEQ ID NO:001) encoded by the pSUPER retrovirus vector comprising the double-stranded DNA segments assembled from SEQ ID NO:002 and SEQ ID NO:003. Oligonucleotide A corresponds to hPit-1a (SEQ ID NO:004) encoded by the pSUPER retrovirus vector comprising the double-stranded DNA segments assembled from SEQ ID NO:002 and SEQ ID NO:003. The control oligonucleotide corresponds to hPit-1c (SEQ ID NO:007) encoded by the pSUPER retrovirus vector comprising the double-stranded DNA segments assembled from SEQ ID NO:008 and SEQ ID NO:009.

FIG. 3 sets forth the effect of Pit-1 siRNA on sodium-dependent phosphate uptake in SMC. Panel A, Phosphate uptake assay was performed by incubation of SMC with buffer containing various concentrations of phosphate for 30 minutes. Data shown are means ±SD (n=3). Panel B, Phosphate uptake was determined by incubation of SMC with buffer containing 0.1 mmol/L phosphate for indicated times. Data shown are means ±SD (n=3). *Significant decrease (P<0.05) compared with SMC-CT.

FIG. 4 sets forth the effect of Pit-1 siRNA on phosphate-induced calcification in SMC. Panel A, SMC-iRNA and SMC-CT were cultured in CM for the indicated times. The calcium content was measured by O-cresolphthalein complexone method and normalized by the protein content. Data shown are means ±SD (n=3). *Significant decrease (P<0.05) compared with SMC-CT. B, SMC-iRNA, SMC-CT, and SMC-mPit1 were cultured in CM for 10 days. After incubation, the cells were fixed with 4% paraformaldehyde. Mineral deposition was visualized by Von Kossa staining. The sections were counterstained with toluidine blue.

FIG. 5 sets forth the effect of Pit-1 overexpression on sodium-dependent phosphate uptake and calcification in SMC. Panel A, RT-PCR analysis of mouse Pit-1 expression. Total RNA was isolated from SMC-mPit1 or SMC-LXIN. PCR was performed with primers for mouse Pit-1 or GAPDH as an internal control. An expected 406-bp fragment of mouse Pit-1 was detected. Panel B, Phosphate uptake was determined by incubation of SMC with 0.1 mmol/L phosphate for 30 minutes as described in Materials and Methods. Data shown are means ±SD (n=3). Panel C, Cells were cultured in CM for 10 days. The calcium content was measured by O-cresolphthalein complexone method and normalized by the protein content. Data shown are means ±SD (n=3). *Significant increase (P<0.05) compared with SMC-LXIN.

FIG. 6 sets forth the characterization of membrane-bound vesicles derived from SMC. Panel A, Matrix vesicles (MV) were isolated from SMC cultures by collagenase digestion and analyzed by transmission electron microscopy. Bar indicates 100 nm. Panel B, SMC were cultured in GM or CM for 2 days; matrix vesicles were prepared by collagenase digestion and were subjected to immunoblot analysis for Annexin V or Pit-1. Unfractionated human SMC lysate was used as a positive control. Panel C, SMC were cultured in SFM or elevated calcium and phosphate containing media (CPM) overnight. Apoptotic bodies (AB) released from cultured SMC were prepared by differentiated centrifugation and subjected to immunoblot analysis for Annexin V or Pit-1. A total of 40 μg of protein was loaded for immunoblot analysis.

FIG. 7 sets forth the assessment of apoptosis in cultured human SMC. Panel A, SMC-iRNA and SMC-CT were cultured in GM or CM for 2 days. Apoptosis was determined by Cell Death Detection ELISA (Roche). As a positive control, SMC apoptosis was induced by treatment with SFM overnight. Data shown are means ±SD (n=3). Panel B, SMC were cultured in GM or CM in the presence or absence of 100 μmol/L of zVAD for 10 days. The calcium content was measured by O-cresolphthalein complexone method and normalized to protein content. Data shown are means ±SD (n=3).

FIG. 8 sets forth the effect of Pit-1 siRNA on induction of Cbfa-1 and OPN by elevated phosphate in SMC. SMC-iRNA and SMC-CT were cultured in GM or CM for seven days. Levels of Cbfa-1 (A) or OPN (B) mRNA were determined by quantitative real-time PCR in triplicate and normalized to 18S rRNA levels. Data are expressed as means ±SD (n=4). *Significant increase (P<0.05) compared with GM.

FIG. 9 sets forth the overexpression of human Pit-2 in SMC-iRNA cells. A full-length human Pit-2 cDNA was inserted into the retroviral vector pBMN-IRES-PURI. Stable Stably-expression of expressing human Pit-2 was established in cells expressing Pit-1 siRNA (SMC-iRNA). Overexpression of Pit-2 was verified by RT-PCR.

FIG. 10 sets forth the restoration of Pi uptake and calcification by restoration of Pit-2 in SMC-iRNA cells. The transport activity of overexpressed Pit-2 was confirmed by Pi uptake assay. Panel A depicts the Pi uptake measured in nmol/mg protein in a 30 min period. Panel B depicts the amount of calcium in ug/mg protein. Overexpression of Pit-2 was able to restore Pi-induced calcification in Pit-1 deficient cells, indicating that Pi uptake by either Pit-1 or Pit-2 is an important determinant of Pi-induced calcification. See Example 9 for experimental details.

FIG. 11 sets forth a pairwise comparison of the nucleotide sequences encoding human Pit-1 and human Pit-2. Identical nucleotide positions are highlighted by an asterisk below the second sequence. The alignment was created using the program Vector NTI 10.1.1 (Invitrogen Inc., Carlsbad, Calif.). The gap opening penalty is 15 and gap extension penalty is 6.66. Gaps are not ignored. The residue fraction for consensus is 0.5, and non-identical residues were considered.

### DETAILED DESCRIPTION OF THE PREFERRED EMBODIMENT

The present invention relates to methods and compositions comprising small interfering RNAs (siRNAs) to identify nucleotides encoding proteins involved in the co-transport of inorganic phosphate in vascular tissues, and the use of these siRNAs as tools to facilitate the identification of proteins involved in vascular calcification, particularly in heart tissues.

Validation that a genetically-controlled factor is involved in the susceptibility of an individual to a disease is critical to the design of treatments which will prevent, ameliorate, treat, or diagnose specific disease conditions. While the medical and pharmaceutical industries have made tremendous progress in the past century developing useful diagnostic tools and therapeutics based on assays designed to determine the properties of small chemical entities, polypeptides, and nucleic acids, the inability to specifically modulate the expression of a test gene or gene product within a cell, tissue, or organism often complicated the interpretation of experiments designed to answer questions relating to the potency and metabolic fate of many compounds.

Recently, the discovery that small double-stranded RNAs (dsRNAs) can be used to facilitate the down-regulation expression of genes in cultured cells or model organisms opened up new avenues for research, greatly accelerating the development of assays used in the drug discovery process. Although the general features of this powerful technology are well known (reviewed in Tuschl, T. Nature Biotechnol. 20: 446-8, 2002; Agrawal et al., Microbiol. Molec. Biol. Rev. 67(4): 657-685, 2003; Kurreck, J., J. Biomedicine Biotechnol. Article ID 83757, pages 1-7), the identification of specific sequences that are necessary to modulate or otherwise knock down the expression of a given gene (encoding a protein), often requires a detailed understanding of factors affecting the temporal (time-related) and cell or tissue-specific expression of a gene or some genetic factor (protein or nucleic acid) controlling expression of a given gene. Briefly, dsRNAs which are between 18 and 30 nucleotides in length which are present in a cell, are able to facilitate the silencing of longer target mRNAs in a process involving hybridization of one of the strands of the dsRNA to the mRNA. The dsRNA is recognized by a cellular endonuclease complex comprising an enzyme called Dicer, or its homologue, that cleaves the mRNA near the end of the duplexed region. Each initial dsRNA (called a silencing RNA, or siRNA) can inactivate multiple copies of the target mRNA. The cleavage products which are not in the regenerated siRNA are rapidly degraded by the cell, as they lack a stabilizing 5′ cap or a 3′ poly(A) tail.

The present invention relates to the identification of Pit-1 and Pit-2 as useful targets in the development methods, compositions, and kits useful in the diagnosis or treatment of diseases characterized by vascular calcification. Preferred embodiments of the invention include methods of identifying siRNAs which specifically target Pit-1 and/or Pit-2, compositions comprising these siRNAs, methods of using the siRNAs in assays to modulate the expression of Pit-1 and/or Pit-2 in cultured cells, isolated tissues, or whole animals as a means to determining the structural characteristics or functional aspects of a test compound in a model system, and as pharmaceutically-acceptable compositions intended for direct administration to an animal or human.

Compounds of the invention include siRNAs which specifically target phosphate cotransporter genes, including Pit-1 and/or Pit-2. Exemplary compounds include siRNAs targeting Pit-1, including those having a specific targeting sequence such as those depicted in SEQ ID NO: 1 and SEQ ID NO:4.

The invention also relates to nucleotides comprising the sequence which specifically target Pit-1 and/or Pit-2 as siRNA, such as those included in SEQ ID NOS:2 and 3, and SEQ ID NOS:5 and 6, which were used in the assembly of plasmid or viral vectors which express the desired siRNAs (SEQ ID NOS:1 and 4, respectively) in a temporal-, cell-, or tissue-specific fashion.

One embodiment of the invention relates to a double-stranded siRNA molecule that specifically down regulates expression of a Pit-1 gene via RNA interference wherein: (a) each strand of said siRNA molecule is independently about 18 to about 28 nucleotides in length; and (b) one strand of said siRNA molecule comprises a target sequence having sufficient complementarity to an RNA of said Pit-1 gene for the siRNA molecule to direct cleavage of the RNA via RNA interference.

In one embodiment of the invention relates to an siRNA molecule that specifically down regulates expression of a Pit-1 gene via RNA interference wherein each strand comprises at least about 14 to about 28 nucleotides that are complementary to the nucleotides of the other strand. Each strand of the siRNA molecule comprises from 19 to 23 nucleotides, and at least one strand of the siRNA molecule has a 3′ overhang of 1 to about 6 nucleotides. Some siRNA molecules may have overhangs of about 2 to 3 nucleotides in length. The siRNA molecules of the invention may comprise molecules wherein the target sequence comprises a nucleotide sequence selected from the group consisting of SEQ ID NO:001 and SEQ ID NO 004. The siRNA molecules of the invention include molecules wherein the target sequence is a sequence unique to Pit-1 mRNA.

The invention includes siRNA molecules defined above, in a pharmaceutically-acceptable carrier or diluent.

Another embodiment of the invention relates to a method of inhibiting expression of Pit-1 in primary, immortal, or transformed cells comprising administering to said cells siRNA molecules that direct cleavage of a target Pit-1 mRNA sequence present in said cells thereby effecting the inhibition. The cells may be selected from the group consisting of smooth muscle cells (SMC), pericytes, valve interstitial cells, osteoblasts, chondrocytes, and mesenchymal stem cells. The siRNA molecules may be about 18 to 28 nucleotides in length, or be about 19 to 23 nucleotides in length. The methods involve siRNA molecules that may comprise two strands, and at least 1 strand of said siRNA molecules has a 3′ overhang of 1 to about 6 nucleotides in length. Some siRNA molecules that have two strands may each have overhangs of about 2 to 3 nucleotides in length.

The methods of the invention also relate to siRNA molecules that comprise molecules wherein the target sequence comprises a nucleotide sequence selected from the group consisting of SEQ ID NO:001 and SEQ ID NO 004. The methods also relate to siRNA molecules wherein the target sequence is a sequence unique to Pit-1 mRNA.

The invention also relates to vectors harboring the specific nucleotides of the invention. An exemplary vector is pSUPER (commercially available from OligoEngine, Seattle, Wash.; Brummelkamp et al. Science 296: 550-553, 2002; US 2003/0144239, which comprises an RNA polymerase III promoter, a region encoding an siRNA, and a transcriptional termination signal comprising five consecutive thymine residues. Other expression vectors based on retroviruses, which permit long term gene silencing, include pRETRO-SUPER (Brummelkamp et al. Cancer Cell, DOI: 10:1016/S1535610802001228, published online: Aug. 22, 2002), pSilencer 3.0-Hi (Ambion) and Block-iT™ Lentiviral Expression System (Invitrogen). Adenoviral expression vector systems can be also used to facilitate the delivery of gene silencing siRNAs. These include the pSilencer Adeno 1.0-CMV vector (Ambion), and the Block-iT™ Adenoviral Expression System (Invitrogen).

Other vectors may be used to facilitate the gene silencing studies of Pit-1 and/or Pit-2. These include the pLXIN retroviral vector (Clontech), pBMN-IRES-PURI (Dr. Garry Nolan, Stanford University), pCI and pSI vectors (Promega), ViraPower T-Rex Lentiviral Expression system (Invitrogen), and pFB-Neo vector (Stratagene).

Chemically synthesized RNA duplexes consisting of two 21 mer oligonucleotides can be transfected into cells using liposomes (such as Lipofectin) or introduced into cells by electroporation for transient gene silencing studies. A variety of companies, including Invitrogen, Ambion, and Qiagen, provide this service. Short 21-23 nt double-stranded siRNA duplexes can be generated by Dicer enzyme in vitro, and then transfected or otherwise introduced into cells directly. Kits are available from several suppliers, including the Silencer siRNA Cocktail Kit (Ambion), the Diced siRNA pools (Invitrogen), and the RNAMaxx@High Yield Transcription Kit (Stratagene).

Other embodiments of the invention include cells comprising the nucleotides of the invention and cells comprising vectors comprising the nucleotides of the invention. Exemplary cell types include smooth muscle cells (SMC), including those derived from human and nonhuman arteries, pericytes, valve interstitial cells, osteoblasts, chondrocytes, and mesenchymal stem cells.

The invention also includes methods of modulating the activity of a Pit-1 or Pit-2 gene in cultured cells, isolated tissues, or whole animals using the nucleotides or vectors of the invention. In one embodiment, a method for determining a modulator of the activity of Pit-1 or Pit-2 comprises determining whether a test agent can modulate the transcription and/or translation of a gene encoding Pit-1 or Pit-2 or the activity of a Pit-1 or Pit-2 polypeptide.

The invention also relates to kits comprising one or more of the following components: polynucleotide compounds of the invention, vectors comprising said polynucleotide compounds, cells comprising said vectors, and/or accessory buffers and reagents needed for detecting and/or quantitating the expression of Pit-1 and/or Pit-2 in a solution, cell, tissue, or organism.

The invention also relates to methods of using the polynucleotide compounds, vectors, cells, or tissues in the manufacture of a medicament for the diagnosis, amelioration, or treatment of a human or animal disease. Preferred diseases include those mediated by disorders of the vascular system, particularly those involving vascular calcification of the arteries.

The invention also includes a method of producing a pharmaceutical composition comprising nucleotides of the invention in a pharmaceutically-acceptable carrier or diluent.

The polynucleotides, vectors, cells, or tissues of the invention may be formulated with standard pharmaceutically-acceptable carriers and/or excipients that are routinely used in the pharmaceutical sciences.

Administration of the medicament is accomplished by any effective route, e.g., orally or parenterally. Methods for parenteral delivery include topical, intra-arterial, subcutaneous, intramedullary, intravenous, or intranasal administration. Oral administration followed by subcutaneous injection would be the preferred routes of uptake; also long acting immobilizations would be used. In addition to the active ingredients, these medicaments may contain suitable pharmaceutically acceptable carriers comprising excipients and other compounds that facilitate processing of the active compounds into preparations which can be used pharmaceutically. Further details on techniques for formulation and administration may be found in the latest edition of “Remington's Pharmaceutical Sciences” (Mack Publishing Co, Easton Pa.).

Medicaments for oral administration can be formulated using pharmaceutically acceptable carriers well known in the art, in dosages suitable for oral administration. Such carriers enable the pharmaceutical compositions to be formulated as tablets, pills, dragees, capsules, liquids, gels, syrups, slurries, suspensions, etc., suitable for ingestion by the patient.

Medicaments for oral use can be obtained through combination of active compounds with solid excipient, optionally grinding a resulting mixture, and processing the mixture of granules, after adding suitable additional compounds, if desired, to obtain tablets or dragee cores. Suitable excipients are carbohydrate or protein fillers. These include, but are not limited to sugars, including lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins, such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage).

Medicaments, which can be used orally, include push-fit capsules made of gelatin, as well as soft, sealed capsules made of gelatin and a coating such as glycerol or sorbitol. Push-fit capsules can contain active ingredients mixed with filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Medicaments for parenteral administration include aqueous solutions of active compounds. For injection, the medicaments of the invention may be formulated in aqueous solutions, preferably in physiologically compatible buffers such as Hank's solution, Ringer's solution, or physiologically buffered saline. Aqueous injection suspensions may contain substances, which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents, which increase the solubility of the compounds to allow for the preparation of highly concentrated solutions.

For topical or nasal administration, penetrants appropriate to the particular barrier to be permeated are used in the formulation. Such penetrants are generally known in the art.

The medicaments of the present invention may be manufactured in a manner similar to that known in the art (e.g., by means of conventional mixing, dissolving, granulating, dragee-making, levigating, emulsifying, encapsulating, entrapping or lyophilizing processes). The medicaments may also be modified to provide appropriate release characteristics, e.g., sustained release or targeted release, by convention means, e.g., coating.

The medicaments may be provided as a salt and can be formed with many acids, including but not limited to hydrochloric, sulfuric, acetic, lactic, tartaric, malic, succinct, etc. Salts tend to be more soluble in aqueous or other protonic solvents that are the corresponding free base forms.

After such medicaments formulated in an acceptable carrier have been prepared, they can be placed in an appropriate container and labeled for treatment of an indicated condition.

Medicaments suitable for prevention or reduction of calcific disease include compositions wherein the active ingredients are contained in an effective amount to achieve the intended purpose. The amount actually administered will be dependent upon the individual to which treatment is to be applied, and will preferably be an optimized amount such that the desired effect is achieved without significant side-effects. The determination of a therapeutically effective dose is well within the capability of those skilled in the art. Of course, the skilled person will realize that divided and partial doses are also within the scope of the invention.

For any compound, the therapeutically effective dose can be estimated initially either in cell culture assays or in any appropriate animal model (e.g., primate, rats and guinea pigs for hypertension and other small laboratory animals). These assays should take into account receptor activity as well as downstream processing activity. The animal model is also used to achieve a desirable concentration range and route of administration. Such information can then be used to determine useful doses and routes for administration in humans.

A therapeutically effective amount refers to that amount of agent, which ameliorates the symptoms or condition. Therapeutic efficacy and toxicity of such compounds can be determined by standard pharmaceutical procedures, in cell cultures or experimental animals (e.g., ED₅₀, the dose therapeutically effective in 50% of the population; and LD₅₀, the dose lethal to 50% of the population). The dose ratio between therapeutic and toxic effects is the therapeutic index, and it can be expressed as the ration ED₅₀/LD₅₀. Medicaments, which exhibit large therapeutic indices, are preferred. The data obtained from cell culture assays and animal studies is used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Long acting medicaments might be administered every 3 to 4 days, every week, or once every two weeks depending on half-life and clearance rate of the particular formulation. Guidance as to particular dosages and methods for delivery is provided in the literature (see U.S. Pat. Nos. 4,657,760; 5,206,344 and 5,225,212, herein incorporated by reference).

The effect of a therapeutic or preventative agent may depend on the underlying cause of the calcific disease, and in some cases it may be preferable to avoid the use of certain treatments for example, the presence or absence of particular alleles of a gene will provide a useful indication as to which is the most appropriate preventative measure.

### EXAMPLES

The foregoing discussion may be better understood in connection with the following representative examples which are presented for purposes of illustrating the principle methods and compositions of the invention and not by way of limitation. Various other examples will be apparent to the person skilled in the art after reading the present disclosure without departing from the spirit and scope of the invention. It is intended that all such other examples be included within the scope of the appended claims.

All parts are by weight, and temperatures are indicated in degrees centigrade (° C.), unless otherwise indicated. Table 1 presents a summary of the of the nucleotide or amino acid sequences described in this application.

TABLE 1
SUMMARY OF SEQUENCES

| | | SEQ |
|---|---|---|
| Nucleotide | | ID |
| name | Sequence | NO |
| human Pit-1b | 5′-GCCTGAAGTATCTCTCCTC-3′ | 001 |
| (Oligo B) | | |
| human Pit-1b | 5′GATCCCCGCCTGAAGTATCTCTCCTCTTCAAGAGAGAGGAGAGAGACTTCAGGCTTTTTGGAAA | 002 |
| (assembly oligo | | |
| B1) | | |
| human Pit-1b | 5′AGCTTTTCCAAAAAGCCTGAAGTCTCTCTCCTCTCTCTTGAAGAGGAGAGATACTTCAGGCGGG | 003 |
| (assembly oligo | | |
| B2) | | |
| human Pit-1a | 5′-TATGTGGCATACCTCTGGA-3′) | 004 |
| (Oligo A) | | |
| human Pit-1a | 5′GATCCCCTATGTGGCATACCTCTGGATTCAAGAGATCCAGAGGCATGCCACATATTTTTGGAAA | 005 |
| (assembly oligo A1) | | |
| human Pit-1a | 5′AGCTTTTCCAAAAATATGTGGCATGCCTCTGGATCTCTTGAATCCAGAGGTATGCCACATAGGG | 006 |
| (assembly oligo | | |
| A2) | | |
| Pit-1c, control | 5′-TCCAGAGGTATGCCACATA-3′ | 007 |
| (Oligo C) | | |
| Pit-1c (assembly | 5′GATCCCCTATGTGGCATGCCTCTGGATTCAAGAGATCCAGAGGTATGCCACATATTTTTGGAAA | 008 |
| oligo C1) | | |
| Pit-1c (assembly | 5′AGCTTTTCCAAAAATATGTGGCATACCTCTGGATCTCTTGAATCCAGAGGCATGCCACATAGGG | 009 |
| oligo C2) | | |
| Human Pit-1 | 5′-TACCATCCTCATCTCGGTGG-3′ | 010 |
| forward | | |
| Human Pit-1 | 5′-TGACGGCTTGACTGAACTGG-3′ | 011 |
| reverse | | |
| Human Pit-1 | t accatcctca tctcggtggg atgtgcagtt ttctgtgccc ttatcgtctg gttctttgta | 012 |
| 410 bp | tgtcccagga tgaagagaaa aattgaacga gaaataaagt gtagtccttc tgaaagcccc ttaatggaaa | |
| PCR fragment | aaaagaatag cttgaaagaa gaccatgaag aaacaaagtt gtctgttggt gatattgaaa acaagcatcc | |
| | tgtttctgag gtagggcctg ccactgtgcc cctccaggct gtggtggagg agagaacagt ctcattcaaa | |
| | cttggagatt tggaggaagc tccagagaga gagaggcttc ccagcgtgga cttgaaagag gaaaccagca | |
| | tagatagcac cgtgaatggt gcagtgcagt tgcctaatgg gaaccttgtc cagttcagtc aagccgtca | |
| Human Pit-2 | 5′-TGGATGGTCATTTTGGGTTT-3′ | 013 |
| forward | | |
| Human Pit-2 | 5′-GCACACCTTTGGTACCGATT-3′ | 014 |
| reverse primer | | |
| Human Pit-2 | tggatg gtcattttgg gtttcatcat agctttcatc ttggcctttt ctgttggtgc aaacgatgtt | 015 |
| 385 bp | gccaactcct ttggtacagc cgtgggctct ggtgtggtga ccttgaggca ggcatgcatt ttagcttcaa | |
| PCR fragment | tatttgaaac caccggctcc gtgttactag gcgccaaagt aggagaaacc attcgcaaag gtatcattga | |
| | cgtgaacctg tacaacgaga cggtggagac tctcatggct ggggaagtta gtgccatggt tggttccgct | |
| | gtgtggcagc tgattgcttc cttcctgagg cttccaatct caggaacgca ctgcattgtg ggttctacta | |
| | taggattctc actggtcgca atcggtacca aaggtgtgc | |
| Mouse Pit-1 | 5′-TACCATCCTCATCTCGGTGG-3′ | 016 |
| forward | | |
| Mouse Pit-1 | 5′-TGACAGTTTGACTGAACTGA-3′ | 017 |
| reverse | | |
| Mouse Pit-1 | taccatcctc atctcggtgg gatgtgcagt tttctgtgcc cttatcgtct | 018 |
| 406 bp | ggttctttgt atgtcccagg atgaagagaa aaattgaacg agaagtaaag tctagtccgt | |
| PCR fragment | ctgaaagtcc cttaatggaa aagaagagca acttaaaaga agaccatgaa gaaacaaaga | |
| | tggctcctgg agacgttgag cataggaatc ctgtgtctga ggtagtgtgt gccactgggc | |
| | cactccgggc tgtggtggag gagaggacgg tgtcattcaa acttggtgac ctggaggagg | |
| | ctccggagcg agagcggctt cccatggacc tgaaggagga gaccagcata gacagcacca | |
| | tcaatggtgc agtgcagttg cctaatggga accttgttca gttcagtcaa actgtca | |
| Human sodium | 5′-GATCCTTCTGGCCTGCCTCA-3′ | 019 |
| hydrogen | | |
| antiporter | | |
| (NHA) forward | | |
| Human sodium | 5′-TCAGGATGGTGCCCAGGTTT-3′ | 020 |
| hydrogen | | |
| antiporter | | |
| (NHA) reverse | | |
| primer | | |
| Human sodium | gatc | 021 |
| hydrogen | cttctggcct gcctcatgaa gataggtttc catgtgatcc ccactatctc aagcatcgtc | |
| antiporter | ccggagagct gcctgctgat cgtggtgggg ctgctggtgg ggggcctgat caagggtgta | |
| PCR fragment | ggcgagacac cccccttcct gcagtccgac gtcttcttcc tcttcctgct gccgcccatc | |
| | atcctggatg cgggctactt cctgccactg cggcagttca cagaaaacct gggcaccatc | |
| | ctga | |
| Full-length | 5′-GGATCCATGGAATCTACTGTGGCAACG-3′ | 022 |
| mouse Pit-1 | | |
| forward primer | | |
| Full-length | 5′-CTCGAGTCACACTGGCAGGATGATGT-3′ | 023 |
| mouse Pit-1 | | |
| reverse primer | | |
| Full-length | 1 atggaatcta ctgtggcaac gattactagt accctagctg ctgttactgc ttccgctcca | 024 |
| mouse | 61 ccgaagtatg acaatctatg gatgctcatc ctgggcttca tcattgcatt tgtcttggca | |
| | 121 ttctccgtgg gagccaatga tgtagcaaat tcgttcggta cagctgtagg ctcaggtgta | |
| | 181 gtgaccctga agcaagcctg catcttagct agcatcttcg aaactgtggg ctccgccttg | |
| | 241 ctgggggcca aagtgagcga aaccatccgg aacggcttga tagatgtgga gctgtacaac | |
| | 301 gaaactcaag atctgctcat ggctggctcc gtcagtgcta tgtttggttc tgctgtgtgg | |
| | 361 cagctcgtgg cttcgttttt gaagcttccg atttctggga cccattgtat tgtcggtgca | |
| | 421 accattggtt tctcccttgt ggcaaatggg cagaagggtg tcaagtggtc tgaactgata | |
| | 481 aaaattgtga tgtcgtggtt cgtctctccg ctgctttctg gtattatgtc tggaatttta | |
| | 541 ttcttccttg ttcgtgcgtt catcctccgt aaggcagatc cggttcctaa tggcttacga | |
| | 601 gctttaccaa ttttttatgc ctgcacaatc ggaatcaacc tcttttccat tatgtatact | |
| | 661 ggagcaccgt tgctgggctt tgacaaactt cctctgtggg gtaccatcct catctcggtg | |
| | 721 ggatgtgcag ttttctgtgc ccttatcgtc tggttctttg tatgtcccag gatgaagaga | |
| | 781 aaaattgaac gagaagtaaa gtctagtccg tctgaaagtc ccttaatgga aaagaagagc | |
| | 841 aacttaaaag aagaccatga agaaacaaag atggctcctg gagacgttga gcataggaat | |
| | 901 cctgtgtctg aggtagtgtg tgccactggg ccactccggg ctgtggtgga ggagaggacg | |
| | 961 gtgtcattca aacttggtga cctggaggag gctccggagc gagagcggct tcccatggac | |
| | 1021 ctgaaggagg agaccagcat agacagcacc atcaatggtg cagtgcagtt gcctaatggg | |
| | 1081 aaccttgttc agttcagtca aactgtcagc aaccagatca actccagtgg ccactatcag | |
| | 1141 tatcacaccg tgcacaagga ttctggcttg tacaaggagc tgctccataa gttacatctg | |
| | 1201 gccaaggtgg gagactgcat gggagattct ggggacaagc ccttgagacg caacaacagc | |
| | 1261 tacacttcct acactatggc aatatgtggc atgcccctgg attcattccg tgccaaagaa | |
| | 1321 ggtgaacaaa agggagatga aatggagacg ctgacatggc ctaatgcaga taccaagaag | |
| | 1381 cggattcgaa tggacagtta caccagttac tgcaatgccg tgtctgacct tcactccgag | |
| | 1441 tctgagatgg acatgagtgt gaaggctgag atgggcctgg gtgacagaaa aggaagcagt | |
| | 1501 ggctctcttg aagaatggta tgaccaggat aagcctgaag tgtcccttct cttccagttc | |
| | 1561 ctgcagatcc ttacagcctg ctttgggtca tttgcccatg gtggcaatga cgtcagcaat | |
| | 1621 gccatcggcc ctctggttgc tttgtatctt gtttataaac aagaagcctc tacaaaagcg | |
| | 1681 gcaacaccca tatggcttct gctttatggt ggtgttggca tttgcatggg cctgtgggtt | |
| | 1741 tggggaagaa gagttatcca gaccatgggg aaggacctga ccccaatcac accctccagt | |
| | 1801 ggtttcagta ttgaactggc gtctgcctta actgtggtca tcgcatcaaa cattggcctt | |
| | 1861 cccatcagca caacacattg caaagtgggc tctgttgtgt ctgttggctg gctccgatca | |
| | 1921 aagaaggctg ttgactggcg actgtttcga aacattttta tggcctggtt tgtcacggtc | |
| | 1981 cccatctctg gggttatcag tgccgctatc atggcagtat tcaagtacat catcctgcca | |
| | 2041 gtgtga | |
| Human Pit-1 | 5′-GGAGGGTGTCAAGTGGTCTGAA-3′ | 025 |
| forward B | | |
| Human Pit-1 | 5′-ATCTGCCTTATGGAGGATGAATG-3′ | 026 |
| reverse B | | |
| Human Pit-1 | 5′-CTGATAAAAATTGTGATGTCTTGG-3′ | 027 |
| the probe B | | |
| Human Pit-2 | 5′-TACAACGAGACGGTGGAGACTCT-3′ | 028 |
| forward B | | |
| Human Pit-2 | 5′-AAGCAATCAGCTGCCACACA-3′ | 029 |
| reverse B | | |
| Human Pit-2 | 5′-CCATGGTTGGTTCCG-3′ | 030 |
| probe B | | |
| Human Cbfa-1 | 5′-CCCGTGGCCTTCAAGGT-3′ | 031 |
| forward | | |
| Human Cbfa-1 | 5′-TGACAGTAACCACAGTCCCATCTG-3′ | 032 |
| reverse | | |
| Human Cbfa-1 | 5′-AGCCCTCGGAGAGGT-3′ | 033 |
| probe | | |
| Human OPN | 5′-TGTCCTCTGAAGAAACCAATGACTT-3′ | 034 |
| forward | | |
| Human OPN | 5′-TCATGGCTTTCGTTGGACTTACT-3′ | 035 |
| reverse | | |
| Human OPN | 5′-AAACAAGAGACCCTTCC-3′ | 036 |
| probe | | |
| Pit-1 | QAVVEERTVSFKLGDLEEAPERERLPSVDLKEETSIDSTV | 037 |
| peptide | | |

Materials and Methods

Human immortalized aortic SMC (human SMC) were used for experiments. SMC stably expressing Pit-1 small interfering double-stranded RNA (siRNA) were generated using the pSUPER RNA interference system (OligoEngine, Seattle, Wash.; Brummelkamp et al. Science 296: 550-553, 2002). Total RNA was isolated from human SMC using the RNeasy kit from Qiagen (Chatsworth, Calif.). Reverse transcription was performed using Omniscript Reverse Transcriptase from Qiagen. TaqMan PCR reagents kits and the ABI Prism 7000 Sequence Detection System (Applied Biosystems, Foster City, Calif.) were used to quantitatively determine levels of human Pit-1, Pit-2, Cbfa-1, and OPN mRNAs. Phosphate uptake assays were performed using H₃³²PO₄. Calcium content was determined using the O-cresolphthalein complexone method. Membrane-bound vesicles and apoptotic bodies were isolated from SMC cultures by differential centrifugation and identified by transmission electron microscopy and biochemical marker analysis.

Cell Culture

Human aortic SMC (human SMC) were isolated, characterized, and immortalized as previously described (Jono et al., Circ Res. 2000; 87:E10-17). Primary human aortic SMC were obtained from Clonetics Corporation (Palo Alto Calif.). Cells were routinely cultured in growth media (GM, DMEM containing 15% FBS, 1.4 mmol/L phosphate, 100 U/mL of penicillin and 100 mg/mL of streptomycin). For elevated phosphate conditions, GM was supplemented with NaH₂PO₄/Na₂HPO₄ to a final concentration of 2.6 mmol/L (calcification media, CM).

Preparation of Stable SMC Expressing Pit-1 Small Interfering RNA (sIRNA)

The pSUPER RNA interference system (OligoEngine, Seattle, Wash.; Brummelkamp et al. Science 296: 550-553, 2002) was used to generate SMC stably expressing Pit-1 siRNA. Initially, we chose two distinct short hairpin RNA (shRNA) sequences (a and b) and one control sequence (c, control) for testing efficiency of knockdown. The shRNA sequences (forward strand and reverse complement) were generated with the sequences below and annealed to create double stranded constructs that were then cloned into pSUPER retrovirus and used to transfect human SMC.

Briefly, an oligonucleotide targeting human Pit-1, designated (hPit-1a), (5′-GCCTGAAGTATCTCTCCTC-3′) (SEQ ID NO:001)

was assembled from the following two complementary oligonucleotides obtained from MWG Biotech Inc (High Point, N.C.):

(SEQ ID NO: 002)
5′-GATCCCCGCCTGAAGTATCTCTCCTCTTCAAGAGAGAGGAGAGAGACTTCAGGCTTTTTGGAAA-3′
(SEQ ID NO: 003)
5′-AGCTTTTCCAAAAAGCCTGAAGTCTCTCTCCTCTCTCTTGAAGAGGAGAGATACTTCAGGCGGG-3′

A second oligonucleotide targeting human Pit-1, designated (hPit-1b), (5′-TATGTGGCATACCTCTGGA-3′) (SEQ ID NO: 004) was assembled from the following two complementary oligonucleotides:

(SEQ ID NO: 005)
5′-GATCCCCTATGTGGCATACCTCTGGATTCAAGAGATCCAGAGGCATGCCACATATTTTTGGAAA-3′
(SEQ ID NO: 006)
5′-AGCTTTTCCAAAAATATGTGGCATGCCTCTGGATCTCTTGAATCCAGAGGTATGCCACATAGGG-3′

A control targeting sequence, designated (hPit-1c, control), (5′-TCCAGAGGTATGCCACATA-3′) (SEQ ID NO:007) was assembled from the following two complementary oligonucleotides:

(SEQ ID NO: 008)
5′-GATCCCCTATGTGGCATGCCTCTGGATTCAAGAGATCCAGAGGTATGCCACATATTTTTGGAAA-3′
(SEQ ID NO: 009)
5′-AGCTTTTCCAAAAATATGTGGCATACCTCTGGATCTCTTGAATCCAGAGGCATGCCACATAGGG-3′

Nucleotides not in bold indicate the Pit-1 target sequence, underlined nucleotides represent single base pair mismatches incorporated in sense strands (hPit-1a and hPit1-b) to facilitate sequence verification, and in antisense strand (control) to generate a control shRNA.

The annealed double-stranded oligonucleotides were inserted into the pSUPER retrovirus vector, and recombinant constructs were transfected into Phoenix packaging cells to generate retrovirus. The infection of SMC was performed using polybrene by exposure to retrovirus for 1 h every 24 h for three consecutive days. The infected cells were selected with 3 μg/ml puromycin for 72 h. SMC containing Pit-1 siRNA are hereafter referred to as SMC-iRNA, whereas the SMC containing the Pit-1 control construct are referred to as SMC-CT.

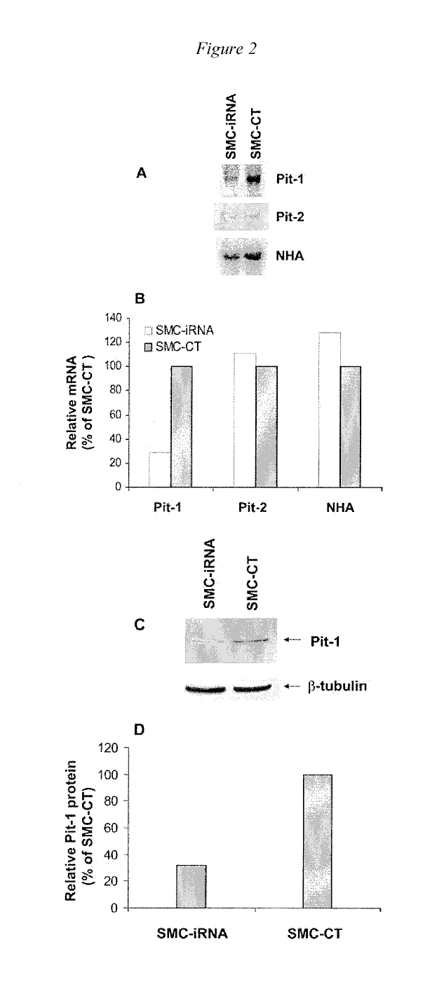

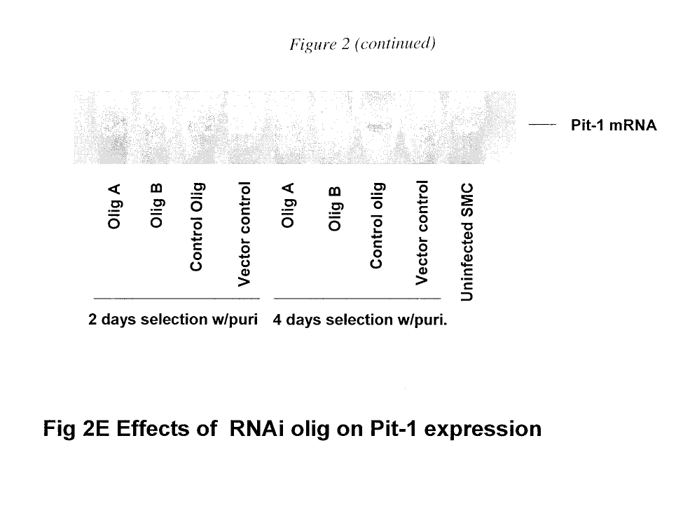

Northern blot analysis revealed that shRNA hPit-1a was more potent for gene silencing than sequence hPit-1b (FIG. 2E). The control sequence had no effect on Pit-1 mRNA levels. Therefore, we chose sequence hPit-1a to make stable Pit-1-deficient cells.

Reverse Transcription PCR and Preparation of cDNA Probes

Total RNA was isolated from cultured SMC using RNeasy kit from Qiagen (Chatsworth, Calif.). Reverse transcription was performed using First-Strand cDNA Synthesis kit (Amersham Biosciences UK, England). The primers used for PCR amplification were:
1) Human Pit-1 forward 5′-TACCATCCTCATCTCGGTGG-3′ (SEQ ID NO:010) and reverse 5′-TGACGGCTTGACTGAACTGG-3′ (SEQ ID NO:011), the 410 bp amplified fragment (SEQ ID NO:012) corresponds to base pairs 1060-1469 of human Pit-1 (L20859).

(SEQ ID NO: 12)

t accatcctca tctcggtggg atgtgcagtt
ttctgtgccc ttatcgtctg gttctttgta tgtcccagga
tgaagagaaa aattgaacga gaaataaagt gtagtccttc
tgaaagcccc ttaatggaaa aaaagaatag cttgaaagaa
gaccatgaag aaacaaagtt gtctgttggt gatattgaaa
acaagcatcc tgtttctgag gtagggcctg ccactgtgcc
cctccaggct gtggtggagg agagaacagt ctcattcaaa
cttggagatt tggaggaagc tccagagaga gagaggcttc
ccagcgtgga cttgaaagag gaaaccagca tagatagcac
cgtgaatggt gcagtgcagt tgcctaatgg gaaccttgtc
cagttcagtc aagccgtca

2) Human Pit-2 forward 5′-TGGATGGTCATTTTGGGTTT-3′ (SEQ ID NO:013) and reverse primer 5′-GCACACCTTTGGTACCGATT-3′ (SEQ ID NO:014), the amplified fragment (SEQ ID NO:015) corresponds to base pairs 265-649 of human Pit-2 (L20852).

(SEQ ID NO: 015)

tggatg gtcattttgg gtttcatcat agctttcatc
ttggcctttt ctgttggtgc aaacgatgtt gccaactcct
ttggtacagc cgtgggctct ggtgtggtga ccttgaggca
ggcatgcatt ttagcttcaa tatttgaaac caccggctcc
gtgttactag gcgccaaagt aggagaaacc attcgcaaag
gtatcattga cgtgaacctg tacaacgaga cggtggagac
tctcatggct ggggaagtta gtgccatggt tggttccgct
gtgtggcagc tgattgcttc cttcctgagg cttccaatct
caggaacgca ctgcattgtg ggttctacta taggattctc
actggtcgca atcggtacca aaggtgtgc

3) Mouse Pit-1 forward 5′-TACCATCCTCATCTCGGTGG-3′ (SEQ ID NO:016) and reverse 5′-TGACAGTTTGACTGAACTGA-3′ (SEQ ID NO:017). The 406 bp PCR product corresponds to position 1111-1517 of mouse Pit-1 (M73696).

SEQ ID NO: 18)

taccatcctc atctcggtgg gatgtgcagt
tttctgtgcc cttatcgtct ggttctttgt atgtcccagg
atgaagagaa aaattgaacg agaagtaaag tctagtccgt
ctgaaagtcc cttaatggaa aagaagagca acttaaaaga
agaccatgaa gaaacaaaga tggctcctgg agacgttgag
cataggaatc ctgtgtctga ggtagtgtgt gccactgggc
cactccgggc tgtggtggag gagaggacgg tgtcattcaa
acttggtgac ctggaggagg ctccggagcg agagcggctt
cccatggacc tgaaggagga gaccagcata gacagcacca
tcaatggtgc agtgcagttg cctaatggga accttgttca
gttcagtcaa actgtca

4) Human sodium hydrogen antiporter (NHA) forward 5′-GATCCTTCTGGCCTGCCTCA-3′ (SEQ ID NO:019) and reverse primer 5′-TCAGGATGGTGCCCAGGTTT-3′ (SEQ ID NO:020), which corresponds to base pairs 377-624 of the human NHA (M81768).

(SEQ ID NO: 21)

gatc
cttctggcct gcctcatgaa gataggtttc catgtgatcc
ccactatctc aagcatcgtc ccggagagct gcctgctgat
cgtggtgggg ctgctggtgg ggggcctgat caagggtgta
ggcgagacac cccccttcct gcagtccgac gtcttcttcc
tcttcctgct gccgcccatc atcctggatg cgggctactt
cctgccactg cggcagttca cagaaaacct gggcaccatc
ctga

The amplified fragments were obtained by RT-PCR from the mRNA of SMC, and then subcloned into the TA cloning vector (Invitrogen, Carlsbad, Calif.) previously described (Jono et al., Circ Res. 2000; 87:E10-17). The identity of the cDNA inserts as Pit-1, Pit-2 or NHA was confirmed by DNA sequence analysis.

Cloning and Expression of Mouse Pit-1

Full-length mouse Pit-1 cDNA was cloned from mouse aortic SMC by RT-PCR, using the forward primer 5′-GGATCCATGGAATCTACTGTGGCAACG-3′ (SEQ ID NO:022) and the reverse primer 5′-CTCGAGTCACACTGGCAGGATGATGT-3′ (SEQ ID NO:023). Sequencing analysis confirmed 100% identity with published mouse Pit-1 sequence.

```
(SEQ ID NO: 024)
      1 atggaatcta ctgtggcaac gattactagt accctagctg ctgttactgc ttccgctcca
    61 ccgaagtatg acaatctatg gatgctcatc ctgggcttca tcattgcatt tgtcttggca
  121 ttctccgtgg gagccaatga tgtagcaaat tcgttcggta cagctgtagg ctcaggtgta
  181 gtgaccctga agcaagcctg catcttagct agcatcttcg aaactgtggg ctccgccttg
  241 ctgggggcca aagtgagcga aaccatccgg aacggcttga tagatgtgga gctgtacaac
  301 gaaactcaag atctgctcat ggctggctcc gtcagtgcta tgtttggttc tgctgtgtgg
  361 cagctcgtgg cttcgttttt gaagcttccg atttctggga cccattgtat tgtcggtgca
  421 accattggtt tctcccttgt ggcaaatggg cagaagggtg tcaagtggtc tgaactgata
  481 aaaattgtga tgtcgtggtt cgtctctccg ctgctttctg gtattatgtc tggaatttta
  541 ttcttccttg ttcgtgcgtt catcctccgt aaggcagatc cggttcctaa tggcttacga
  601 gctttaccaa ttttttatgc ctgcacaatc ggaatcaacc tcttttccat tatgtatact
  661 ggagcaccgt tgctgggctt tgacaaactt cctctgtggg gtaccatcct catctcggtg
  721 ggatgtgcag ttttctgtgc ccttatcgtc tggttctttg tatgtcccag gatgaagaga
  781 aaaattgaac gagaagtaaa gtctagtccg tctgaaagtc ccttaatgga aaagaagagc
  841 aacttaaaag aagaccatga agaaacaaag atggctcctg gagacgttga gcataggaat
  901 cctgtgtctg aggtagtgtg tgccactggg ccactccggg ctgtggtgga ggagaggacg
  961 gtgtcattca aacttggtga cctggaggag gctccggagc gagagcggct tcccatggac
1021 ctgaaggagg agaccagcat agacagcacc atcaatggtg cagtgcagtt gcctaatggg
1081 aaccttgttc agttcagtca aactgtcagc aaccagatca actccagtgg ccactatcag
1141 tatcacaccg tgcacaagga ttctggcttg tacaaggagc tgctccataa gttacatctg
1201 gccaaggtgg gagactgcat gggagattct ggggacaagc ccttgagacg caacaacagc
1261 tacacttcct acactatggc aatatgtggc atgcccctgg attcattccg tgccaaagaa
1321 ggtgaacaaa agggagatga aatggagacg ctgacatggc ctaatgcaga taccaagaag
1381 cggattcgaa tggacagtta caccagttac tgcaatgccg tgtctgacct tcactccgag
1441 tctgagatgg acatgagtgt gaaggctgag atgggcctgg gtgacagaaa aggaagcagt
1501 ggctctcttg aagaatggta tgaccaggat aagcctgaag tgtcccttct cttccagttc
1561 ctgcagatcc ttacagcctg ctttgggtca tttgcccatg gtggcaatga cgtcagcaat
1621 gccatcggcc ctctggttgc tttgtatctt gtttataaac aagaagcctc tacaaaagcg
1681 gcaacaccca tatggcttct gctttatggt ggtgttggca tttgcatggg cctgtgggtt
1741 tggggaagaa gagttatcca gaccatgggg aaggacctga ccccaatcac accctccagt
1801 ggtttcagta ttgaactggc gtctgcctta actgtggtca tcgcatcaaa cattggcctt
1861 cccatcagca caacacattg caaagtgggc tctgttgtgt ctgttggctg gctccgatca
1921 aagaaggctg ttgactggcg actgtttcga aacattttta tggcctggtt tgtcacggtc
1981 cccatctctg gggttatcag tgccgctatc atggcagtat tcaagtacat catcctgcca
2041 gtgtga
```

The PCR product containing SEQ ID NO:24 was cloned into the EcoRI site of the retroviral expression vector pLXIN (BD Biosciences, Palo Alto, Calif.). Recombinant pLXIN containing the mouse Pit-1 cDNA or empty vector were transfected into the Phoenix packaging cell line by calcium-phosphate precipitation to generate retrovirus. Retroviral infection of SMC-iRNA was performed as described above. The transduced cells containing mouse Pit-1 or empty vector were referred to as SMC-mPit1 or SMC-LXIN respectively. Pooled populations of cells were used for experiments.

Quantitative Real-Time PCR

Total RNA was isolated from human SMC using the RNeasy kit from Qiagen (Chatsworth, Calif.). Reverse transcription was performed using Omniscript Reverse Transcriptase from Qiagen. Levels of human Pit-1, Pit-2, Cbfa-1 and OPN mRNAs were determined by quantitative real-time PCR performed with TaqMan PCR reagents kits in the ABI Prim 7000 Sequence Detection System (Applied Biosystems, Foster City, Calif.). The primers and probes were designed using Primer-Express software V2.0 (Applied Biosystems). The primers were synthesized by Qiagen and the Taq probes were obtained from the Applied Biosystems. All of the Taq probes have a FAM (fluorochrome reporter) tag at the 5′-end and an MGB quencher at the 3′-end and the sequences are:

1)
Human Pit-1 forward

(SEQ ID NO:025)

5′-GGAGGGTGTCAAGTGGTCTGAA-3′
and
reverse

(SEQ ID NO:026)

5′-ATCTGCCTTATGGAGGATGAATG-3′,
the probe

(SEQ ID NO:027)

5′-CTGATAAAAATTGTGATGTCTTGG-3′.
2)
Human Pit-2 forward

(SEQ ID NO:028)

5′-forward-TACAACGAGACGGTGGAGACTCT-3′
and
reverse

(SEQ ID NO:029)

5′-AAGCAATCAGCTGCCACACA-3′,
the probe

(SEQ ID NO:030)

5′-CCATGGTTGGTTCCG-3′.
3)
Human Cbfa-1 forward

(SEQ ID NO:031)

5′-CCCGTGGCCTTCAAGGT-3′
and
reverse

(SEQ ID NO:032)

5′-TGACAGTAACCACAGTCCCATCTG-3′,
the probe

(SEQ ID NO:033)

5′-AGCCCTCGGAGAGGT-3′.
4)
Human OPN forward

(SEQ ID NO:034)

5′-TGTCCTCTGAAGAAACCAATGACTT-3′
and
reverse

(SEQ ID NO:035)

5′-TCATGGCTTTCGTTGGACTTACT-3′,
the probe

(SEQ ID NO:036)

5′-AAACAAGAGACCCTTCC-3′.

All PCR reactions were performed in triplicate. Quantification of gene expression was calculated by the standard curve method according to the manufacturer's protocol and normalized to 18S rRNA.

Northern Blot Analysis

Total RNA was isolated from SMC with Trizol (Invitrogen Inc., Carlsbad, Calif.). Total RNA was separated by electrophoresis, transferred to a Zeta-probe blotting membrane (Bio-Rad Laboratories, Hercules, Calif.), and hybridized with ³²P-labeled cDNA probes for human Pit-1, Pit-2 or NHA as previously described (Jono et al., Circ Res. 2000; 87:E10-17). Relative mRNA levels were quantified by densitometric scanning and normalized to 18S rRNA. The sequence of the 18S rRNA probe was described previously (Steitz et al., Circ Res. 2001; 89: 1147-1154).

Phosphate Uptake Assays

Phosphate uptake assays were performed as previously described (Jono et al., Circ Res. 2000; 87:E10-17). In brief, SMC were incubated in Earle's buffered salt solution (EBSS) containing various concentrations of phosphate (including H₃³²PO₄ obtained from PerkinElmer Life Science, Inc. Boston, Mass.). Sodium-dependent phosphate uptake was determined by subtracting uptake in the presence of EBSS containing choline chloride from uptake in EBSS containing sodium chloride. Uptake values were normalized to cellular protein content.

Calcium Quantitation

SMC calcification was induced by the treatment with calcification media (CM, 2.6 mmol/L phosphate). Calcium content of the cultures was determined using the O-cresolphthalein complexone method exactly as previously described (Jono et al., Circ Res. 2000; 87:E10-17) and normalized to protein content.

Western Blot Analysis

Cell lysates were prepared as described previously (Steitz et al., Circ Res. 2001; 89:1147-1154). Equal amounts of protein were loaded and separated by 8% SDS-PAGE followed by transfer to PVDF membrane. The membranes were then blotted using anti-Pit-1 or anti-Annexin V antibodies (R&D System, Minneapolis, Minn.). Pit-1 antibody was obtained by immunizing rabbits with the Pit-1 peptide,

QAVVEERTVSFKLGDLEEAPERERLPSVDLKEETSIDSTV (SEQ ID NO:037), followed by peptide affinity purification of serum using Sulfo-Link kit from Pierce Biotechnology, Inc. (Rockford, Ill.). Specific proteins were detected with enhanced chemiluminescencereagents (Perkin Elmer Life Science, Inc. Boston, Mass.).

Membrane-Bound Vesicle Isolation

Membrane-bound, extracellular vesicles were prepared by two different methods as described previously (Montessuit et al., J Bone Miner Res. 1995; 10:625-631; Reynolds et al., J Am Soc Nephrol. 2004; 15:28572867). For extracellular matrix-derived vesicles, after 2 day incubation with normal growth media (GM, containing 1.4 mmol/L phosphate) or calcification media (CM, containing 2.6 mmol/L phosphate), SMC monolayers were washed and then incubated with 500 U/ml of collagenase for 3 hours at 37° C. The digest was centrifuged for 20 mins at 15,000 g to pellet cell debris. The supernatant was centrifuged for 60 min at 100,000 g. The resulting pellet containing extracellular matrix-derived vesicles was resuspended and used for subsequent experiments. Alternatively, SMC were incubated overnight with either serum-free media (SFM, serum-free GM supplemented with 0.5% BSA) or elevated calcium and phosphate containing media (CPM, serum-free media with 2.7 mmol/L calcium and 2.0 mmol/L phosphate). The culture medium was collected and centrifuged for 20 min at 2500 rpm to remove the debris, the supernatant (containing shed membrane-bound vesicles) was then spun for 30 min at 100,000 g.

Apoptosis Assay

The Cell Death Detection ELISA kit (Roche Diagnostics Co, Indianapolis, Ind.) was used according to the manufacturer's direction to quantitatively determine cytoplasmic histone-associated DNA oligonucleosome fragments associated with apoptotic cell death (Castigli et al., Am J Physiol Cell Physiol. 2000; 279:C2043-2049).

Statistical Analysis

Results are expressed as mean ±SD. Significance between groups was determined by ANOVA, p-values less than 0.05 were considered significant.

### Example 1

### Pit-1 is the Predominant Sodium-Dependent Phosphate Cotransporter Expressed in Human SMC

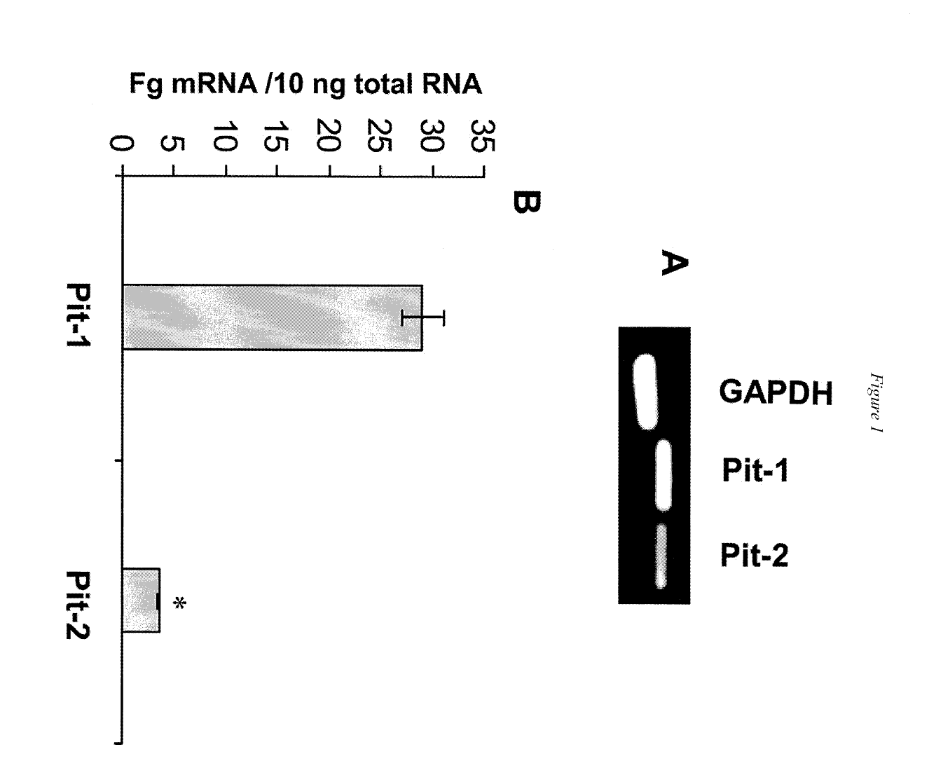

Our previous studies indicated that sodium-dependent phosphate cotransporters might be involved in phosphate-induced SMC calcification (Jono et al., Circ Res. 2000; 87: e10-e17). To determine the expression profile of sodium-dependent phosphate cotransporters in human SMC, the abundance of type I, type II, and type III sodium-dependent phosphate cotransporters were determined initially using RT-PCR. Using this technique, no bands were obtained using primers for NPT1 (human type I family) and NaPi-3 (human type II family) (data not shown). However, a strong band at 409 bp was obtained using Pit-1 primers, and a weaker band at 384 bp was amplified using Pit-2 primers (FIG. 1A), indicating that members of the human type III family were present. Sequence analysis of these amplified fragments confirmed their identities as human Pit-1 and Pit-2, respectively (data not shown). Similar results were obtained in primary human aortic SMC (data not shown). Real-time PCR was used to precisely quantitate the expression levels of Pit-1 and Pit-2 in the cells. As shown in FIG. 1B, the Pit-1 mRNA levels were 8-fold higher than that of Pit-2 in human SMC. These results indicated that Pit-1 is the predominant form of sodium-dependent phosphate cotransporter expressed in human SMC. Therefore, subsequent experiments focused on determining the role of Pit-1 in human SMC calcification.

### Example 2

### Downregulation of Pit-1 mRNA Leads to Decreased Pit-1 Protein Levels in Human SMC

Previous studies using phosphonoformic acid, a generic sodium-dependent phosphate cotransporter inhibitor, implicated a crucial role for these cotransporters in phosphate-mediated SMC calcification (Jono et al., Circ Res. 2000; 87: e10-e17). To more specifically address the role of Pit-1 in this process, RNA interference was used to suppress endogenous Pit-1 mRNA levels. SMC stably expressing Pit-1 siRNA (SMC-iRNA) or control construct (SMC-CT) were established using a retroviral system. Effects on Pit-1 expression levels were examined by Northern and Western blot analysis. As shown in FIGS. 2A and 2B, Pit-1 mRNA levels were dramatically reduced (80%) in SMC-iRNA compared with levels in SMC-CT. Western blotting confirmed a comparable decrease in Pit-1 protein levels in SMC-iRNA (FIGS. 2C and 2D).

To determine the specificity of the Pit-1 siRNA, mRNA levels of 2 other membrane transporters, Pit-2 and sodium hydrogen antiporter (NHA), were measured. Pit-2 is the other member of type III sodium-dependent phosphate cotransporter family and shares 58% homology at the amino acid level with Pit-1 (Miller et al., Proc Natl Acad Sci USA. 1994; 91: 78-82). Despite this sequence similarity, Pit-1 siRNA had no effect on Pit-2 mRNA levels. Similarly, NHA mRNA was not inhibited by Pit-1 siRNA (FIGS. 2A and 2B). These results demonstrated that the Pit-1 siRNA specifically inhibited Pit-1 expression.

### Example 3

### Inhibition of Sodium-Dependent Phosphate Uptake by Pit-1 siRNA

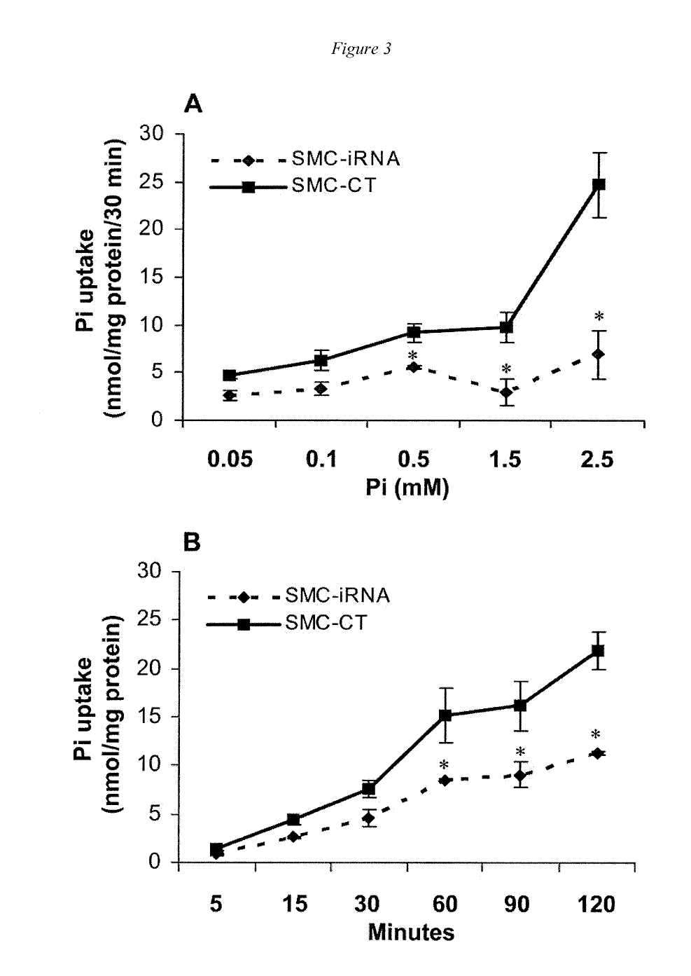

To determine whether decreased Pit-1 mRNA and protein levels translated to decreased Pit-1 function, phosphate uptake assays were performed in SMC-iRNA and SMC-CT (as noted in the general methods section of the Examples, noted above). As shown in FIGS. 3A and 3B, phosphate uptake was concentration and time-dependent in both cell types. However, overall phosphate uptake was substantially decreased in SMC-iRNA compared with SMC-CT at all concentrations (at 30 minutes in 1.5 mmol/L Pi, SMC-iRNA versus SMC-CT: 2.90 versus 9.78 nmol/mg protein, respectively). Likewise, phosphate uptake was substantially decreased in SMC-iRNA compared with SMC-CT at all time points (at 120 minutes in 0.1 mmol/L Pi, SMC-iRNA versus SMC-CT: 11.27 versus 21.90 nmol/mg protein, respectively). These results indicated that a decrease in cellular Pit-1 levels caused by the Pit-1 siRNA resulted in a comparable decrease in phosphate uptake. No effect on sodium-independent phosphate transport was observed (data not shown).

### Example 4

### Inhibition of SMC Calcification by Pit-1 siRNA

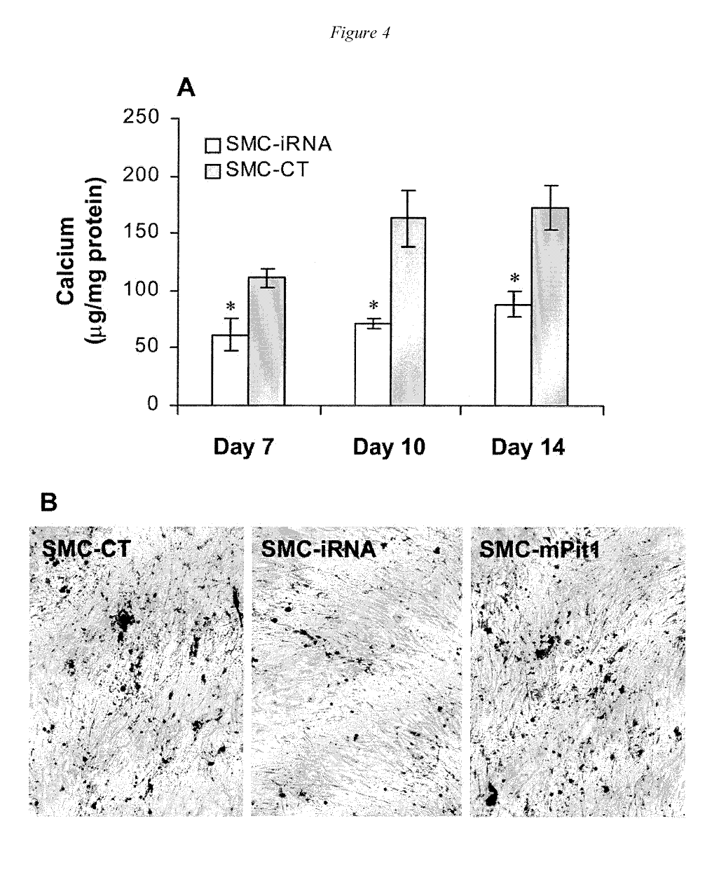

To determine whether Pit-1 knockdown affected the ability of human SMC to calcify, both SMC-iRNA and SMC-CT were incubated with either growth media (GM) (1.4 mmol/L phosphate) or calcification media (CM) (2.6 mmol/L phosphate) for 7, 10, and 14 days, and calcification was determined biochemically (by the O-cresolphthalein complexone method, noted above) and histologically as shown in FIG. 4A. SMC-iRNA showed significantly less calcium deposition than SMC-CT at all time points (at day 10, SMC-iRNA versus SMC-CT: 71.84 versus 163.09 μg/mg protein, respectively). Mineral deposits were further identified morphologically by Von Kossa staining and light microscopy. As shown in FIG. 4B, after culture in CM for 10 days, abundant mineral deposits associated with extracellular matrix could be detected throughout the SMC-CT culture. Comparable to biochemical data, much less mineral deposition was observed in SMC-iRNA compared with SMC-CT. No mineral deposits were observed in cultured SMC treated with GM at any time points (data not shown). These results suggested that Pit-1 was necessary for SMC calcification induced by elevated phosphate in vitro.

### Example 5

### Restoration of Calcification by Overexpression of Mouse Pit-1 in Pit-1-Deficient Human SMC

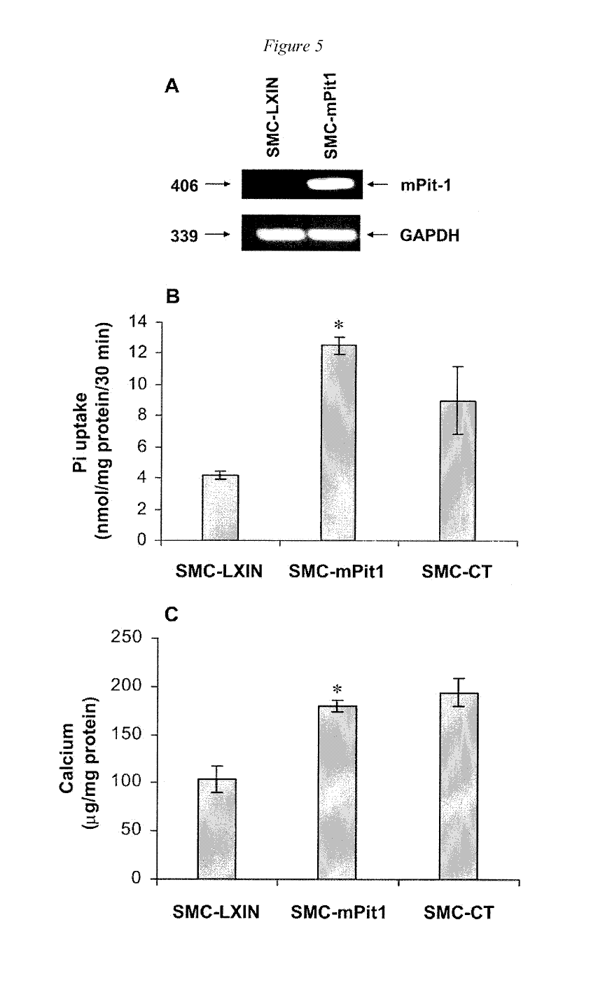

To further determine the specific requirement of Pit-1 in SMC calcification and to rule out potential off-target effects of Pit-1 siRNA, we examined whether overexpression of Pit-1 could restore phosphate-induced calcification in SMC-iRNA. To do this, we took advantage of a 3-bp mismatch between human and mouse Pit-1 in the siRNA targeting region. Thus, the human Pit-1 siRNA is unable to target the mouse Pit-1 transcript for degradation. Retroviral constructs encoding mouse Pit-1 cDNA or empty vector pLXIN were, therefore, generated and used to infect SMC-iRNA. The transduced cells are referred to as SMC-mPit1 or SMC-LXIN, respectively. As shown in FIG. 5A, a 406-bp fragment for mouse Pit-1 was amplified in SMC-mPit1. No amplified fragment was detected in SMC-LXIN. Next we examined the functionality of the expressed mouse Pit-1 by performing phosphate uptake assays. FIG. 5B shows that phosphate uptake was increased 3-fold in SMC-mPit1 compared with SMC-LXIN (at 30 minutes in 0.1 mmol/L Pi, SMC-mPit1 versus SMC-LXIN: 12.51 versus 4.18 nmol/mg protein, respectively). The phosphate uptake in SMC-mPit1 was comparable to that of SMC-CT. These results demonstrated that the overexpressed mouse Pit-1 functioned properly as a phosphate transporter in SMC-iRNA.

We next determined whether overexpression of Pit-1 restored calcification induced by elevated phosphate. SMC-mPit1 or SMC-LXIN and SMC-CT were cultured in CM for 10 days, and calcification was determined biochemically and histologically. As shown in FIG. 5C, calcium deposition was almost twice as high in SMC-mPit1 compared with SMC-LXIN (SMC-mPit1 versus SMC-LXIN: 179.84 versus 103.44 μg/mg protein, respectively). Furthermore, the calcium content in SMC-mPit1 was almost the same as SMC-CT (SMC-mPit1 versus SMC-CT: 179.84 versus 194.14 μg/mg protein, respectively). Von Kossa staining revealed that overexpression of mPit-1 increased the formation of mineral deposits in the extracellular matrix of cultured SMC, and the levels of calcification in these cells were comparable to that of SMC with normal Pit-1 levels (FIG. 4B). Taken together, these results suggest that Pit-1 is an important mediator of calcification in SMC.

### Example 6

### Pit-1 is not a Component of Matrix Vesicles or Apoptotic Bodies Derived from SMC

One possible mechanism for the requirement of Pit-1 in SMC calcification is in mediating phosphate loading of calcifying extracellular vesicles. Growing evidence suggests that membrane-bound vesicles derived from cells may be involved in both physiological and pathological calcification. In bone and cartilage, matrix vesicles have been identified as cell-derived, membrane-bound vesicles intimately associated with sites of active extracellular matrix mineralization (Anderson H C, Curr Rheumatol Rep. 2003; 5: 222-226). In addition, Shanahan and colleagues have described calcifying membrane-bound vesicles and apoptotic bodies released from human aortic SMC cultured in serum-free media (SFM) (Reynolds et al., J Am Soc Nephrol. 2004; 15: 2857-2867). In both cases, it is postulated that these membrane-bound vesicles participate in mineralization by concentrating calcium and phosphate in a protected microenvironment, thereby promoting mineral nucleational events.

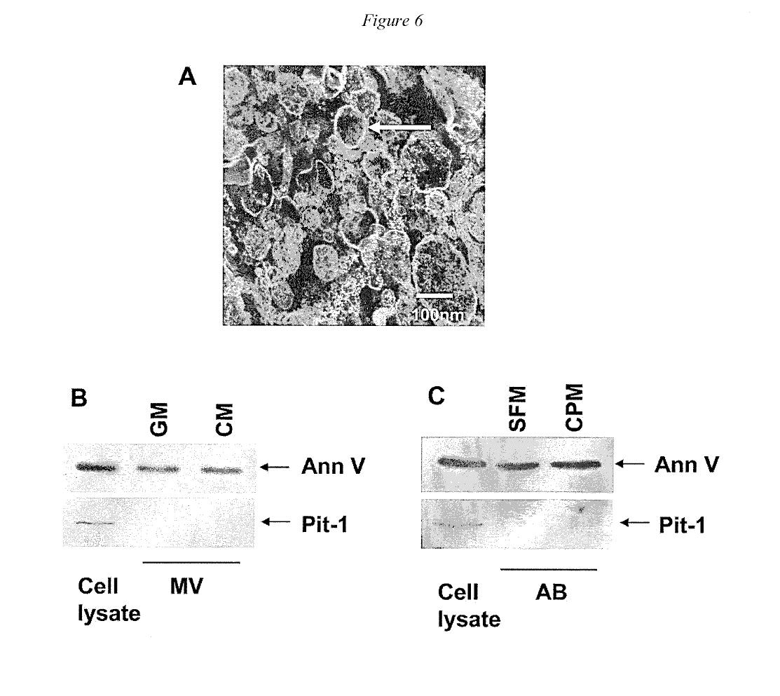

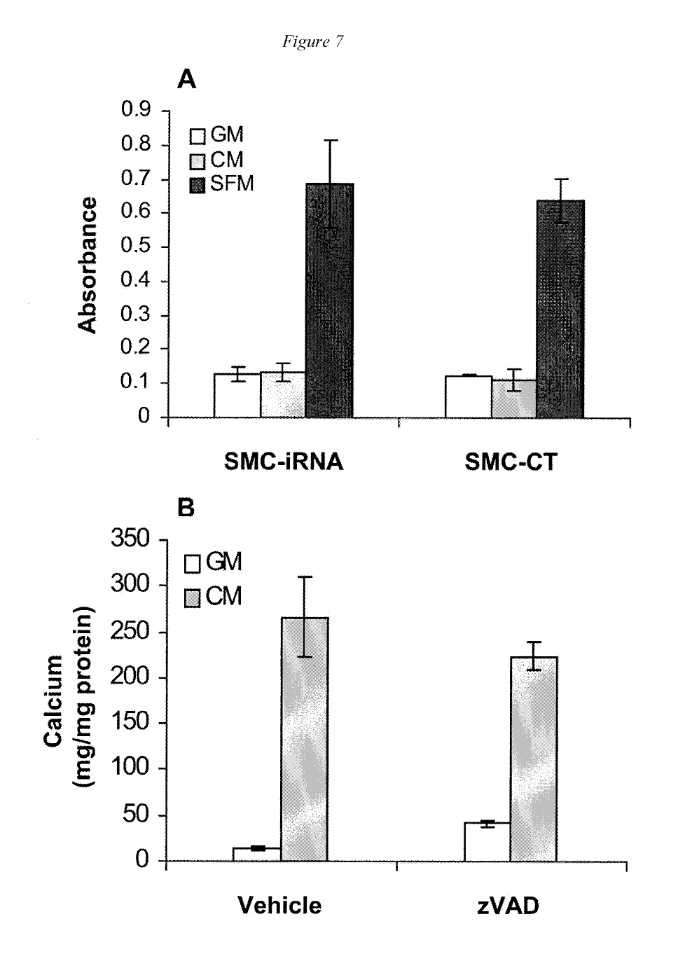

To determine whether Pit-1 was a component of SMC-derived vesicles, we used two different methods to isolate membrane-bound vesicles from cultured SMC. In the first approach, the classic collagenase method for isolation of matrix vesicles from bone and cartilage was used (Montessuit et al., J Bone Miner Res. 1995; 10: 625-631). As shown in FIG. 6A, transmission electron microscopy revealed that vesicles derived from the extracellular matrix of human SMC were membrane bound and heterogeneous in shape and size, with diameters ranging from 100 to 200 nm, identical to those described from chondrocyte cultures (D'Angelo et al., J Biol. Chem. 2001; 276: 11347-11353). Equivalent amounts of matrix vesicles, as determined by protein content, were isolated from SMC cultured in CM compared with in GM (CM versus GM: 2.9 versus 3.0 mg/mL protein respectively). Western blot analysis demonstrated that these vesicles contained Annexin V (FIG. 6B), a previously identified marker of matrix vesicles (Anderson H C. Curr Rheumatol Rep. 2003; 5: 222-226). Annexin V levels were identical in matrix vesicles isolated from human SMC cultured in GM or CM, suggesting again that elevated phosphate treatment does not alter matrix vesicle formation or protein content. However, no Pit-1 protein was detected in these matrix vesicle preparations under either normal or elevated phosphate conditions (FIG. 6B), suggesting that Pit-1 was not involved in phosphate loading by these structures.

In the second approach, membrane-bound vesicles and apoptotic bodies were isolated from apoptotic human SMC following serum starvation under normal (SFM) or elevated calcium and phosphate conditions (CPM), as previously described (Reynolds et al., J Am Soc Nephrol. 2004; 15: 2857-2867). Identical to results obtained with matrix vesicles, Pit-1 was not detected in these apoptotic vesicle preparations under any condition examined (FIG. 6C).

### Example 7

### Pit-1 does not Promote SMC Apoptosis in Response to Elevated Phosphate

Previous studies have shown that apoptosis can accelerate mineralization in serum-deprived and long-term cultured human SMC (Reynolds et al., J Am Soc Nephrol. 2004; 15: 2857-2867; Proudfoot et al., Circ Res. 2000; 87: 1055-1062). Thus, another possible mechanism for elevated phosphate- and Pit-1-mediated SMC calcification might be through stimulation of apoptosis. To determine whether elevated phosphate induced SMC death via Pit-1, we examined apoptosis in SMC-iRNA and SMC-CT cells. Both cells were cultured in GM or CM and apoptosis was detected by specific ELISA to monitor mono- and oligonucleosomal chromatid formation. As shown in FIG. 7A, very little apoptosis was detected in both cell types when cultured in GM. Importantly, treatment of the SMC-CT cells with CM did not increase apoptosis, and identical results were obtained in SMC-iRNA cells. As expected, treatment of cells with SFM overnight strongly induced apoptosis, and levels were similar in SMC-CT and SMC-iRNA cells (FIG. 7A). Finally, treatment of human SMC with 100 μmol/L zVAD, a potent caspase inhibitor, did not inhibit elevated phosphate-induced calcification (FIG. 7B). These results indicate that the phosphate concentrations used in the present studies do not stimulate SMC apoptosis and that cells deficient in Pit-1 (SMC-iRNA) are not less susceptible to cell death than cells containing normal levels of Pit-1 (SMC-CT). Thus, apoptosis is unlikely the mechanism for Pit-1 mediated SMC calcification.

### Example 8

### Pit-1 is Required for Elevated Phosphate-Induced Osteogenic Gene Expression in Human SMC

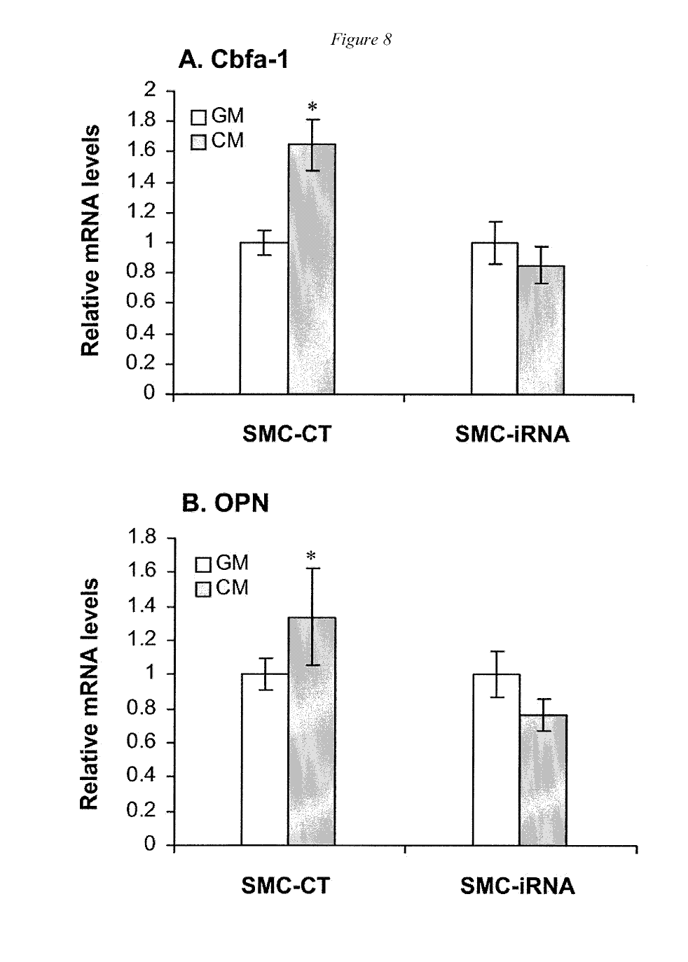

We and others have previously shown that elevated phosphate stimulates SMC calcification concomitant with phenotypic change, characterized by expression of osteogenic genes such as Cbfa-1 and OPN (Steitz et al., Circ Res. 2001; 89: 1147-1154; Chen et al., Kidney Int. 2002; 62: 1724-1731; Jono et al., Phosphate regulation of vascular smooth muscle cell calcification. Circ Res. 2000; 87: e10-e17). To determine whether Pit-1 was required for elevated phosphate-induced SMC phenotypic transition, we examined Cbfa-1 and OPN mRNA levels in SMC-CT and SMC-iRNA using real-time PCR. As expected, both Cbfa-1 and OPN mRNA levels were significantly induced by elevated phosphate relative to normal phosphate conditions in SMC-CT at 7 days following treatment (FIGS. 8A and 8B). In contrast, Cbfa-1 and OPN mRNA levels did not increase in response to elevated phosphate treatment compared with normal phosphate conditions in SMC-iRNA. Similar results were obtained when SMC-CT and SMC-iRNA were incubated with elevated phosphate for 2 days (data not shown). These results suggest that Pit-1 was required for expression of Cbfa-1 and OPN mRNA in human SMC in response to elevated phosphate conditions.

### DISCUSSION

Previous studies indicated that elevated phosphate could induce SMC calcification as well as an osteochondrogenic phenotypic change in vitro (Steitz et al., Circ Res. 2001; 89: 1147-1154; Jono et al., Circ Res. 2000; 87: e10-e17). Blocking the activity of sodium-dependent phosphate cotransporters with the nonspecific inhibitor phosphonoformic acid inhibited SMC calcification, suggesting an important role for phosphate uptake in SMC calcification. In the present study, we examined the requirement for the major sodium-dependent phosphate cotransporter in SMC, Pit-1, in calcification using RNA interference and overexpression approaches. Stable expression of Pit-1 siRNA specifically inhibited Pit-1 mRNA and protein levels and led to a dramatic decrease in sodium-dependent phosphate uptake in SMC. Calcification, in response to elevated phosphate, was substantially inhibited in SMC stably expressing Pit-1 siRNA. Restoration of phosphate uptake by overexpression of Pit-1 in Pit-deficient SMC rescued phosphate-induced calcification. Elevated phosphate-induced SMC phenotypic transition, as exemplified by upregulation of Cbfa-1 and OPN mRNA levels, was Pit-1 dependent. These results demonstrate that Pit-1, via phosphate uptake, plays a critical role in human SMC calcification in response to elevated phosphate.

The present studies confirm that vascular calcification is a highly cell-regulated process. Several local and systemic, cell-derived inhibitors of calcification, such as osteopontin, pyrophosphate, and matrix gla protein, whose deficiency leads to inappropriate vascular calcification in vivo and in vitro have recently been identified (Rutsch et al., Am J Pathol. 2001; 158: 543-554; Speer et al., Cardiovasc Res. 2005; 66: 324-333; Speer et al., J Exp Med. 2002; 196: 1047-1055; Luo et al., Nature. 1997; 386: 78-81). A growing number of positive regulators of calcification, such as lipids, cytokines, and elevated phosphate levels, have also been identified (Demer L L, Int J. Epidemiol. 2002; 31: 737-741). Induction of SMC calcification by elevated phosphate is of particular interest, because hyperphosphatemia is a major risk factor for vascular calcification in dialysis patients. In the current studies, modulation of the levels of a cell membrane transporter, Pit-1, controlled the susceptibility of SMC to calcification in the presence of elevated phosphate. Despite equivalent calcium and phosphate levels in the culture media, cells that were deficient in Pit-1 had substantially reduced extracellular matrix calcification compared with cells with normal levels of Pit-1. These findings strongly suggest that cellular mechanisms, including phosphate uptake and/or signaling via Pit-1 is required for SMC calcification.

There is considerable evidence suggesting the important role of matrix vesicles in the mineralization of bone and cartilage by concentrating calcium and phosphate and initiating mineral nucleation (Anderson H C, Curr Rheumatol Rep. 2003; 5: 222-226). A recent study by Shanahan and colleagues identified membrane-bound vesicles containing apoptotic bodies from apoptotic human SMC that could calcify after prolonged exposure to elevated calcium or phosphate (Reynolds et al., J Am Soc Nephrol. 2004; 15: 2857-2867). Although matrix vesicles as well as apoptotic bodies could be isolated from human SMC in the present study, we were unable to detect Pit-1 protein in either preparation, regardless of culture under normal or elevated phosphate conditions. Equivalent numbers of matrix vesicles and apoptotic bodies were isolated in normal and elevated phosphate containing media. Thus, it is unlikely that Pit-1 mediates calcification of human SMC by facilitating synthesis, release, or phosphate loading of matrix vesicles or apoptotic bodies.

Several studies have suggested that extremely high phosphate levels (4 to 10 mmol/L) induced apoptosis in chondrocytes as well as SMC and that mineralization was mediated, in part, through apoptosis-dependent mechanisms (Reynolds et al., J Am Soc Nephrol. 2004; 15: 2857-2867; Proudfoot et al., Circ Res. 2000; 87: 1055-1062; Magne et al., J Bone Miner Res. 2003; 18: 1430-1442; Mansfield et al., Bone. 2001; 28: 1-8). In the current studies, human SMC cultured in serum-containing media in the presence of 2.6 mmol/L phosphate did not show increased apoptosis compared with cells cultured in normal phosphate containing media (1.4 mmol/L phosphate). Cells deficient in Pit-1 had very low levels of apoptosis that were comparable to cells with normal Pit-1 levels, and the susceptibility of these cells to apoptosis was not altered by elevated phosphate treatment. The caspase inhibitor zVAD also failed to prevent phosphate-induced calcification in our system. These results suggest that phosphate-induced apoptosis is not the mechanism by which elevated phosphate induces SMC calcification under our culture conditions. These findings are in contrast to Reynolds et al., who showed that elevated calcium (2.7 mmol/L) combined with elevated phosphate (2.0 mmol/L) induced apoptosis as well as calcification of human SMC cultured in SFM (Reynolds et al., J Am Soc Nephrol. 2004; 15: 2857-2867). The difference in these findings is most likely explained by differences in culture conditions. Reynolds et al., used SFM combined with elevated calcium and phosphate to induce SMC apoptosis, apoptotic body formation, and calcification, whereas the human SMC in our studies were cultured in serum-containing media, with calcium and phosphate levels kept well below concentrations that could result in spontaneous mineral precipitation in solution or cause apoptosis. Incubation of human SMC in SFM also strongly induced apoptosis in our study. Thus, the requirement of Pit-1 in human SMC calcification under the more physiological conditions of our system does not appear to be through a mechanism involving altered apoptosis.

Human SMC deficient in Pit-1 failed to upregulate Cbfa-1 and OPN mRNA levels in response to elevated phosphate treatment. Both genes were induced in human SMC containing normal levels of Pit-1, consistent with previous findings (Steitz et al., Circ Res. 2001; 89: 1147-1154; Jono et al., Circ Res. 2000; 87: e10-e17). These results suggest that phosphate uptake through Pit-1 is required for the phenotypic modulation of human SMC to an osteochondrocytic phenotype, thereby regulating the mineralization capacity of these cells.

Pit-1 has also been implicated in bone cell differentiation and mineralization. Pit-1 mRNA increases during osteoblast differentiation and correlates with the time at which calcification is observed (Nielsen et al., Bone. 2001; 28: 160-166). Studies by Palmer et al. found that Pit-1 mRNA was expressed in mineralizing, hypertrophic chondrocytes from day 17 in embryonic murine metatarsals, whereas no Pit-1 mRNA was detected in fully differentiated, nonmineralized chondrocytes (Palmer et al., Bone. 1999; 24: 1-7). Thus, phosphate uptake via Pit-1 may be a major regulator of cellular differentiation programs involved in both normal and pathological calcification.

### Example 9

### Rescue of PI Transport Function in Pit-1 Knockdown Cells by Pit-2 Expression

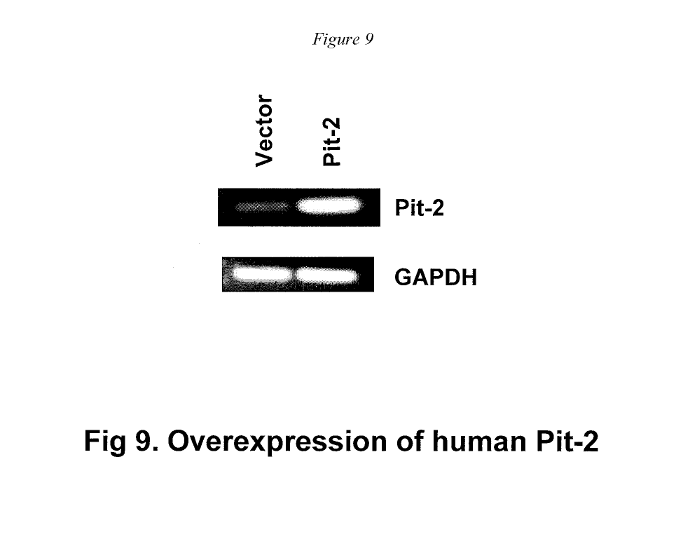

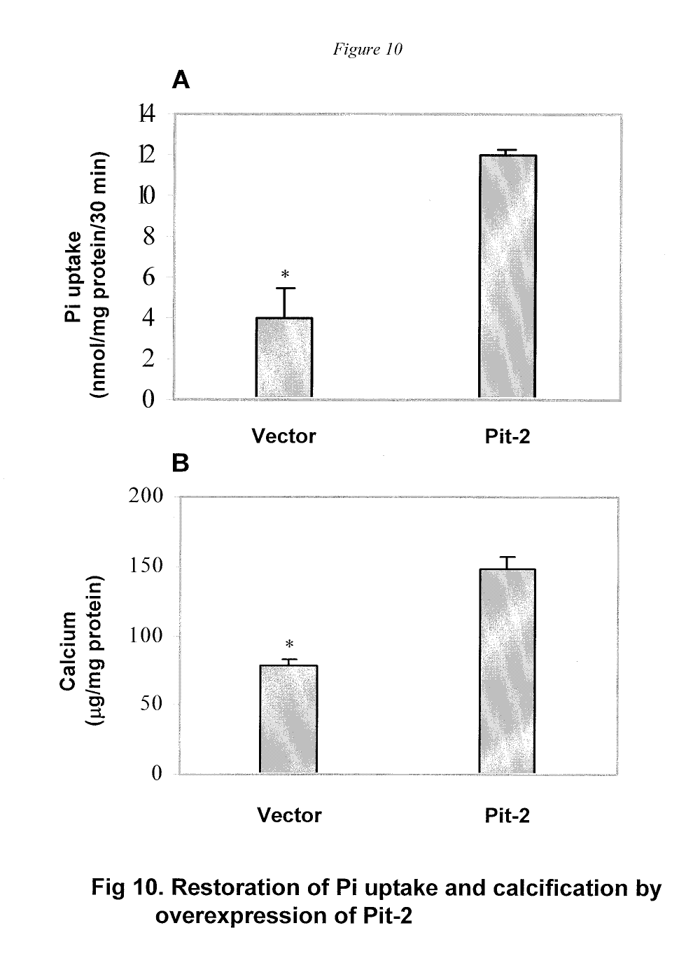

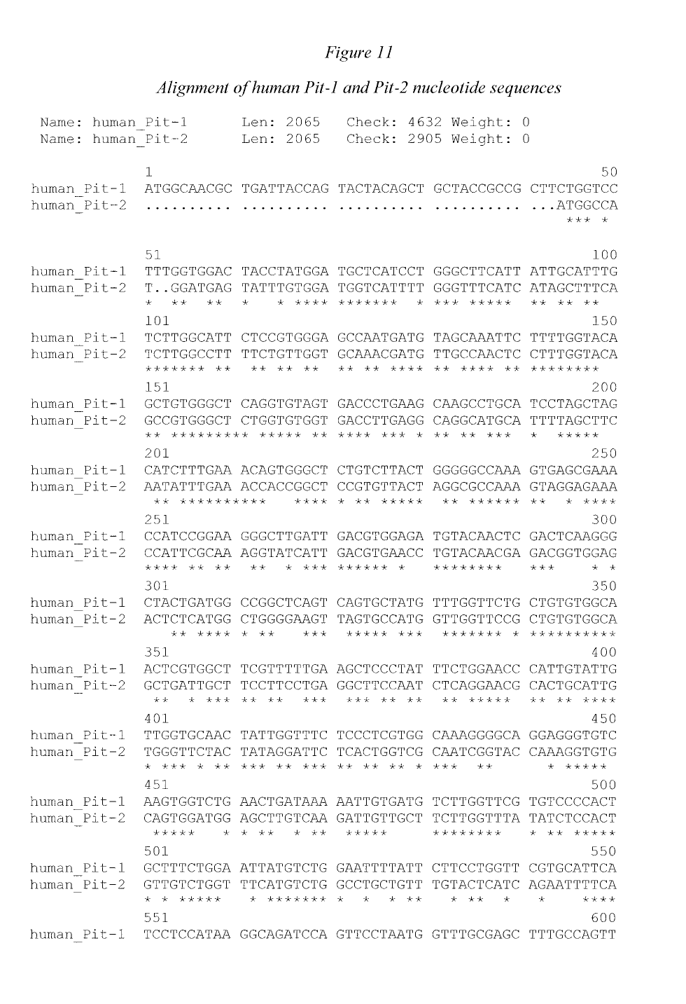

Full-length human Pit-2 cDNA (obtained from Dr. Jean Heart, Institute Pasteur, Paris) was inserted into pBMN-IRES-PURI (obtained from Dr. Garry Nolan, Stanford University) to make a recombinant retroviral construct. The virus generated from the packaginc cell line was used to infect Pit-1-siRNA cells. The infected cells over express Pit-2 protein and therefore, restore Pi transport function in Pit-1 knockdown cells. Overexpression of Pit-2 was verified by RT-PCR (FIG. 9). The transport activity of overexpressed Pit-2 was confirmed by Pi uptake assay. In addition, overexpression of Pit-2 was able to restore Pi-induced calcification in Pit-1 deficient cells (FIG. 10), indicating that Pi uptake by either Pit-1 or Pit-2 is an important determinant of Pi-induced calcification.

### Example 10

### Identification of Pit-2 siRNAS

Pit-2 siRNAs are identified by gene walking or the generation of Pit-2 specific siRNA libraries using methods described by Seyhan et al., (RNA 11: 837-846, 2005). Vascular calcification and SMC phenotype transition are determined by the methods described above for Pit-1 siRNAs. Pit-2 siRNAs are used to treat human SMC.

Other siRNAs that potently and specifically target Pit-1 and Pit-2 are identified using a gene walk strategy whereby overlapping 22 bp sequences encompassing the entire Pit-1 or Pit-2 mRNAs are screened in appropriate cells (Rocchi P et al., BJU Int. 2006 November; 98(5):1082-1089). Alternatively, efficacious siRNAs can be identified using gene-specific siRNA libraries (Seyhan, A A. et al., RNA 11: 837-846, 2005).

### Example 11

### Genetically Engineered Mice Containing a Floxed Pit-1 Gene

Mice containing loxp sequences flanking exon 2-4 of the Pit-1 gene are generated, and crossed with a deleter mouse that expresses Cre recombinase in all tissues to generate a global Pit-1 null mouse, or with tissue specific Cre recombinase expressing mouse to generate a conditional Pit-1 knockout mouse. For example, crossing the floxed Pit-1 mouse with the SM22-Cre recombinase transgenic mouse generates a mouse in which Pit-1 is deleted specifically in smooth muscle cells. These mice are used to examine the role of Pit-1 in mouse models of vascular calcification, including that caused by chronic renal failure, atherosclerosis, or diabetes. Similar studies can be done conditionally targeting Pit-2. The conditional knockouts are used to examine the mechanism of vascular calcification and to validate upstream and downstream targets of Pit-1 and Pit-2.

### Example 12

### Comparison of Human Pit-1 and Pit-2 Nucleotide Sequences

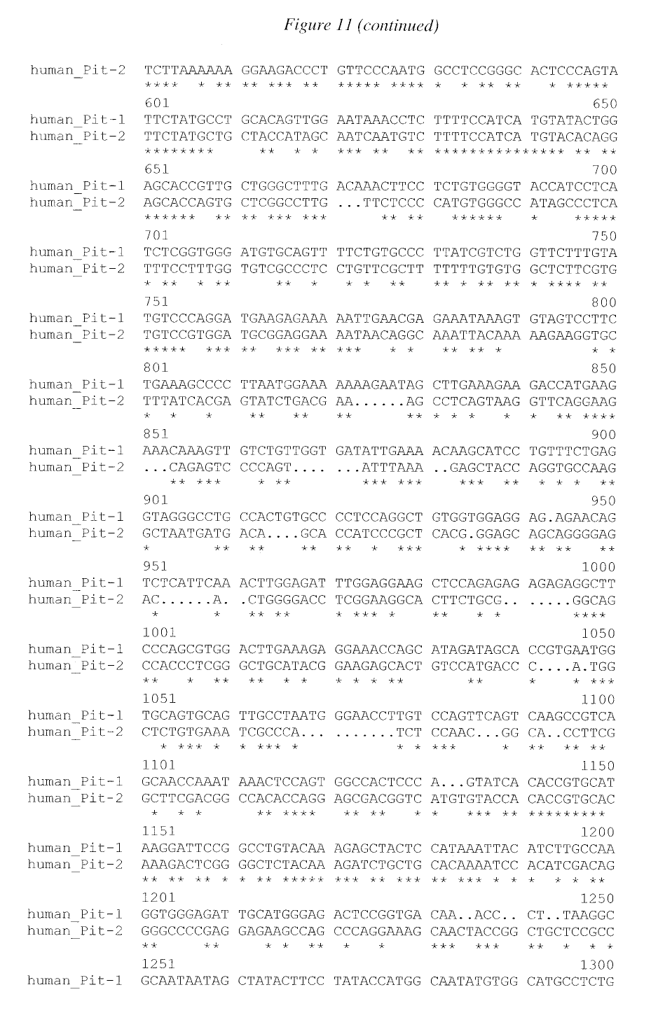

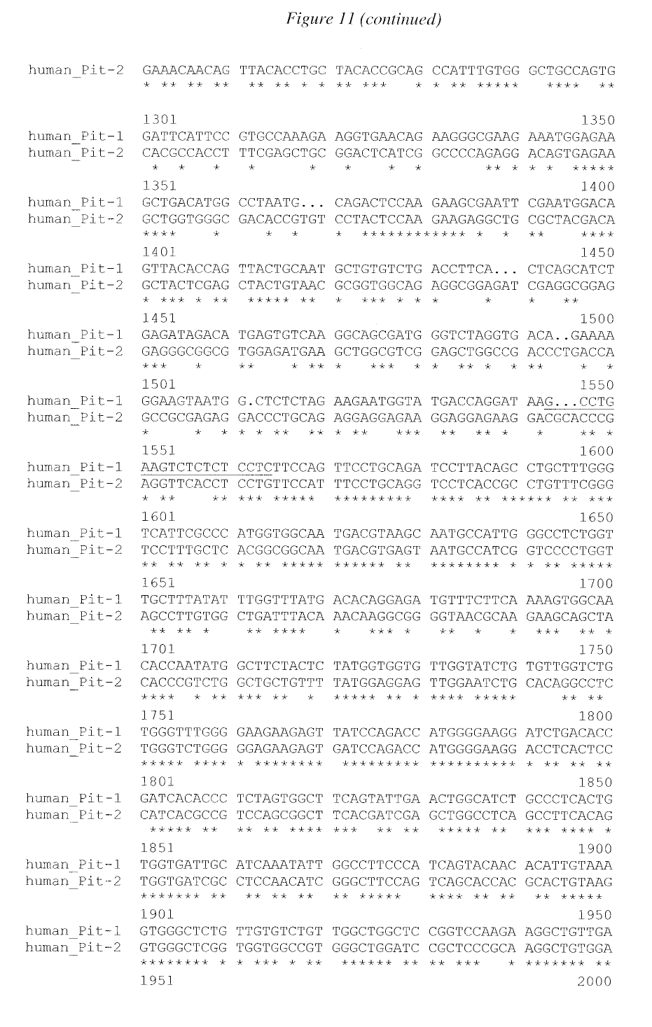

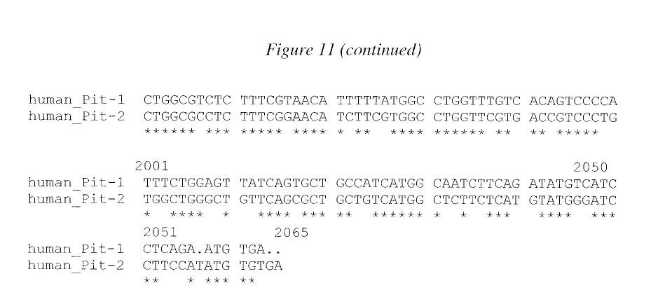

The nucleotide sequences encoding human Pit-1 and Pit-2 were compared to determine if there were any stretches of identical nucleotides that would be suitable for preparing single siRNA molecules that would be effective in targeting both Pit-1 and Pit-2. As shown in FIG. 11, there are no regions that appear to be sufficiently identical that would be useful in designing siRNAs that target both genes.

While the preferred embodiment of the invention has been illustrated and described, it will be appreciated that various changes can be made therein without departing from the spirit and scope of the invention.

### REFERENCES

All references, patents, or applications cited herein are incorporated by reference in their entirety, as if written herein.
Agrawal, N, Dasaradhi, P V N, Mohmmed, A, Malhotra, P, Bhatnagar, R K, Mukherjee, S K. RNA interference: biology, mechanism, and applications. Microbiol. Molec. Biol. Rev. 67(4): 657-685, 2003.
Alfrey A C, Ibels L S. Role of phosphate and pyrophosphate in soft tissue calcification. Adv Exp Med. Biol. 1978; 103: 187-193.
Anderson H C. Matrix vesicles and calcification. Curr Rheumatol Rep. 2003; 5: 222-226.
Block G A, Hulbert-Shearon T E, Levin N R, Port F K. Association of serum phosphorus and calcium x phosphate product with mortality risk in chronic hemodialysis patients: a national study. Am J Kidney Dis. 1998; 31: 607-617.
Brummelkamp, T R, Bernards, R, Agami, R. A system for stable expression of short interfering RNAs in mammalian cells. Science 296: 550-553, 2002.
Brummelkamp, T R, Bernards, R, Agami, R. Stable suppression of tumorigenicity by virus-mediated RNA interference. Cancer Cell, DOI: 10:1016/S1535610802001228, Published online: Aug. 22, 2002.
Castigli E, Arcuri C, Giovagnoli L, Luciani R, Secca T, Gianfranceschi G L, Bocchini V. Interleukin-1beta induces apoptosis in GL15 glioblastoma-derived human cell line. Am J Physiol Cell Physiol. 2000; 279:C2043-2049.
Chen N X, O'Neill K D, Duan D, Moe S M. Phosphorus and uremic serum up-regulate osteopontin expression in vascular smooth muscle cells. Kidney Int. 2002; 62: 1724-1731.
Christian R C, Fitzpatrick L A. Vascular calcification. Curr Opin Nephrol Hypertens. 1999; 8: 443-448.
D'Angelo M, Billings P C, Pacifici M, Leboy P S, Kirsch T. Authentic matrix vesicles contain active metalloproteases (MMP). A role for matrix vesicle-associated MMP-13 in activation of transforming growth factor-beta. J Biol. Chem. 2001; 276: 11347-11353.
Demer L L. Vascular calcification and osteoporosis: inflammatory responses to oxidized lipids. Int J Epidemiol. 2002; 31: 737-741.
Giachelli C M. Vascular calcification: in vitro evidence for the role of inorganic phosphate. J Am Soc Nephrol. 2003; 14: S300-S304.
Goodman W G, Goldin J, Kuizon B D, Yoon C, Gales B, Sider D, Wang Y, Chung J, Emerick A, Greater L, Elashoff R M, Salusky I B. Coronary-artery calcification in young adults with end-stage renal disease who are undergoing dialysis. N Engl J. Med. 2000; 342:1478-1483.
Jono S, McKee M D, Murry C E, Shioi A, Nishizawa Y, Mori K, Morii H, Giachelli C M. Phosphate regulation of vascular smooth muscle cell calcification. Circ Res. 2000; 87: e10-e17.
Jono S, McKee M D, Murry C E, Shioi A, Nishizawa Y, Mori K, Morii H, Giachelli C M. Phosphate regulation of vascular smooth muscle cell calcification. Circ Res. 2000; 87:E10-17.
Kavanaugh M P, Kabat D. Identification and characterization of a widely expressed phosphate transporter/retrovirus receptor family. Kidney Int. 1996; 49: 959-963.
Kurreck, J. siRNA efficiency: structure or sequence—that is the question. J. Biomedicine Biotechnol. Article ID 83757, pages 1-7.
London G M, Guerin A P, Marchais S J, Metivier F, Pannier B, Adda H. Arterial media calcification in end-stage renal disease: impact on all-cause and cardiovascular mortality. Nephrol Dial Transplant. 2003; 18: 1731-1740.
London G M, Pannier B, Marchais S J, Guerin A P. Calcification of the aortic valve in the dialyzed patient. J Am Soc Nephrol. 2000; 11: 778-783.
Luo G, Ducy P, McKee M D, Pinero G J, Loyer E, Behringer R R, Karsenty G. Spontaneous calcification of arteries and cartilage in mice lacking matrix GLA protein. Nature. 1997; 386: 78-81.
Magne D, Bluteau G, Faucheux C, Palmer G, Vignes-Colombeix C, Pilet P, Rouillon T, Caverzasio J, Weiss P, Daculsi G, Guicheux J. Phosphate is a specific signal for ATDC5 chondrocyte maturation and apoptosis-associated mineralization: possible implication of apoptosis in the regulation of endochondral ossification. J Bone Miner Res. 2003; 18:1430-1442.
Mansfield K, Teixeira C C, Adams C S, Shapiro I M. Phosphate ions mediate chondrocyte apoptosis through a plasma membrane transporter mechanism. Bone. 2001; 28: 1-8.
Miller D G, Edwards R H, Miller A D. Cloning of the cellular receptor for amphotropic murine retroviruses reveals homology to that for gibbon ape leukemia virus. Proc Natl Acad Sci USA. 1994; 91: 78-82.
Montessuit C, Bonjour J P, Caverzasio J. Expression and regulation of Na-dependent P(i) transport in matrix vesicles produced by osteoblast-like cells. J Bone Miner Res. 1995; 10: 625-631.
Montessuit C, Bonjour J P, Caverzasio J. Expression and regulation of Na-dependent P(i) transport in matrix vesicles produced by osteoblast-like cells. J Bone Miner Res. 1995; 10:625-631.
Nielsen L B, Pedersen F S, Pedersen L. Expression of type III sodium-dependent phosphate transporters/retroviral receptors mRNAs during osteoblast differentiation. Bone. 2001; 28:160-166.
O'Hara B, Johann S V, Klinger H P, Blair D G, Rubinson H, Dunn K J, Sass P, Vitek S M, Robins T. Characterization of a human gene conferring sensitivity to infection by gibbon ape leukemia virus. Cell Growth Differ. 1990; 1: 119-127.
Palmer G, Zhao J, Bonjour J, Hofstetter W, Caverzasio J. In vivo expression of transcripts encoding the Glvr-1 phosphate transporter/retrovirus receptor during bone development. Bone. 1999; 24: 1-7.
Proudfoot D, Skepper J N, Hegyi L, Bennett M R, Shanahan C M, Weissberg P L. Apoptosis regulates human vascular calcification in vitro: evidence for initiation of vascular calcification by apoptotic bodies. Circ Res. 2000; 87: 1055-1062.
Raggi P, Boulay A, Chasan-Taber S, Amin N, Dillon M, Burke S K, Chertow G M. Cardiac calcification in adult hemodialysis patients. A link between end-stage renal disease and cardiovascular disease? J Am Coll Cardiol. 2002; 39: 695-701.
Reynolds J L, Joannides A J, Skepper J N, McNair R, Schurgers L J, Proudfoot D, Jahnen-Dechent W, Weissberg P L, Shanahan C M. Human vascular smooth muscle cells undergo vesicle-mediated calcification in response to changes in extracellular calcium and phosphate concentrations: a potential mechanism for accelerated vascular calcification in ESRD. J Am Soc Nephrol. 2004; 15: 2857-2867.
Rocchi, P, et al., Small interference RNA targeting heat-shock protein 27 inhibits the growth of prostatic cell lines and induces apoptosis via caspase-3 activation in vitro. BJU Int. 2006 November; 98(5):1082-9.
Rutsch F, Vaingankar S, Johnson K, Goldfine I, Maddux B, Schauerte P, Kalhoff H, Sano K, Boisvert W A, Superti-Furga A, Terkeltaub R. PC-1 nucleoside triphosphate pyrophosphohydrolase deficiency in idiopathic infantile arterial calcification. Am J. Pathol. 2001; 158: 543-554.
Seyhan, A A, et al., Complete, gene-specific siRNA libraries: Production and expression in mammalian cells. RNA 11: 837-846 2005
Shigematsu T, Kono T, Satoh K, Yokoyama K, Yoshida T, Hosoya T, Shirai K. Phosphate overload accelerates vascular calcium deposition in end-stage renal disease patients. Nephrol Dial Transplant. 2003; 18 (suppl 3): iii86-iii89.
Speer M Y, Chien Y C, Quan M, Yang H Y, Vali H, McKee M D, Giachelli C M. Smooth muscle cells deficient in osteopontin have enhanced susceptibility to calcification in vitro. Cardiovasc Res. 2005; 66: 324-333.
Speer M Y, Giachelli C M. Regulation of cardiovascular calcification. Cardiovasc Pathol. 2004; 13: 63-70.
Speer M Y, McKee M D, Guldberg R E, Liaw L, Yang H Y, Tung E, Karsenty G, Giachelli C M. Inactivation of the osteopontin gene enhances vascular calcification of matrix Gla protein-deficient mice: evidence for osteopontin as an inducible inhibitor of vascular calcification in vivo. J Exp Med. 2002; 196: 1047-1055.
Steitz S A, Speer M Y, Curing a G, Yang H Y, Haynes P, Aebersold R, Schinke T, Karsenty G, Giachelli C M. Smooth muscle cell phenotypic transition associated with calcification: upregulation of Cbfa1 and downregulation of smooth muscle lineage markers. Circ Res. 2001; 89: 1147-1154.
Sugitani H, Wachi H, Murata H, Sato F, Mecham R P, Seyama Y. Characterization of an in vitro model of calcification in retinal pigmented epithelial cells. J Atheroscler Thromb. 2003; 10: 48-56.
Takeda E, Taketani Y, Morita K, Miyamoto K. Sodium-dependent phosphate co-transporters. Int J Biochem Cell Biol. 1999; 31: 377-381.
Tuschl, T. Expanding small RNA interference. Nature Biotechnol. 20: 446-8, 2002.
United States Renal Data System. USRDS1999 Annual Data Report. Bethesda, Md.: National Institutes of Health, National Institute of Diabetes and Digestive and Kidney Disease; 1999. Available at: www.usrds.org/chapters/ch06.pdf. Accessed Dec. 10, 2002.
Wada T, McKee M D, Steitz S, Giachelli C M. Calcification of vascular smooth muscle cell cultures: inhibition by osteopontin. Circ Res. 1999; 84: 166-178.
Wayhs R, Zelinger A, Raggi P. High coronary artery calcium scores pose an extremely elevated risk for hard events. J Am Coll Cardiol. 2002; 39: 225-230.
Werner A, Dehmelt L, Nalbant P. Na+-dependent phosphate cotransporters: the NaPi protein families. J Exp Biol. 1998; 201: 3135-3142.
Wilson P W, Kauppila L I, O'Donnell C J, Kiel D P, Hannan M, Polak J M, Cupples L A. Abdominal aortic calcific deposits are an important predictor of vascular morbidity and mortality. Circulation. 2001; 103: 1529-1534.

The embodiments of the invention in which an exclusive property or privilege is claimed are defined as follows:

1. A double-stranded siRNA molecule that specifically down regulates expression of a Pit-1 gene via RNA interference wherein:
(a) each strand of said siRNA molecule is independently about 18 to about 28 nucleotides in length; and
(b) one strand of said siRNA molecule comprises a target sequence having sufficient complementarity to an RNA of said Pit-1 gene for the siRNA molecule to direct cleavage of the RNA via RNA interference.

2. The siRNA molecule of claim 1 wherein each strand comprises at least about 14 to about 28 nucleotides that are complementary to the nucleotides of the other strand.

3. The siRNA molecule of claim 2 wherein each strand of said siRNA molecules comprised from 19 to 23 nucleotides.

4. The siRNA molecule of claim 3 wherein at least 1 strand of the siRNA molecule has a 3′ overhang of 1 to about 6 nucleotides in length.

5. The siRNA molecule of claim 4 wherein both strands of the siRNA molecule have overhangs of about 2 to 3 nucleotides in length.

6. The siRNA molecule of claim 1 wherein said target sequence comprises a nucleotide sequence selected from the group consisting of SEQ ID NO:001 and SEQ ID NO:004.

7. The method of claim 1 wherein the target sequence is a sequence unique to Pit-1 mRNA.

8. A composition comprising the siRNA molecule of any of claims 1-3 in a pharmaceutically-acceptable carrier or diluent.
